(19) United States
(12) Patent Application Publication
(10) Pub. No.: US 2026/0183563 A1
(43) Pub. Date: Jul. 2, 2026

(54) WEARABLE DEVICE FOR THE DELIVERY OF FOCUSSED LIGHT INTO THE BODY

(71) Applicant: The Trustees of Columbia University in the City of New York, New York, NY (US)

(72) Inventors: Kenneth Shepard, New York, NY (US); Nanfang Yu, New York, NY (US); Brett Youngerman, New York, NY (US)

(73) Assignee: The Trustees of Columbia University in the City of New York, New York, NY (US)

(21) Appl. No.: 19/438,843

(22) Filed: Jan. 2, 2026

Related U.S. Application Data
(60) Provisional application No. 63/741,299, filed on Jan. 2, 2025.

(51) Int. Cl. A61N 5/06; A61N 5/067; H01S 5/00; H01S 5/125; H01S 5/343; H01S 5/42

(52) U.S. Cl. CPC A61N 5/062; A61N 5/0622; A61N 5/067; H01S 5/0085; H01S 5/125; H01S 5/423; A61N 2005/0627; A61N 2005/0645; A61N 2005/065; A61N 2005/0658; A61N 2005/0663; A61N 2005/0665; H01S 5/3432; H01S 5/3434

(57) ABSTRACT

An exemplary wearable device for a focused direction of a light into a tissue and a method for method for providing such wearable device can be provided, such exemplary device can comprise an array of laser devices, and a configuration that can include metasurfaces and/or spatial light modulators (SLMs). the exemplary metasurfaces and/or the spatial light modulators can facilitate a wavefront shaping for each of the laser devices.

### CROSS-REFERENCE TO RELATED APPLICATIONS

This application claims the benefit of U.S. Provisional Application No. 63/741,299 filed Jan. 2, 2025, the contents of which is hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### BACKGROUND OF THE INVENTION

Scattering (primarily Rayleigh scattering with 1/λ⁴ wavelength dependence) along with absorption is the principal impediment to getting light to depths exceeding 15 mm into tissue and controlling its spatial extent. For light into the head, the mean-free path (λ_{mfp}) is about 1.4 mm in the scalp, leading to almost completely diffusive transport by 4 mm into the scalp.

Thus, there is a need to address and/or improve various issues and/or deficiencies which exist in the previous devices, systems, and processes.

The following is intended to be a brief summary of the exemplary embodiments of the present disclosure, and is not intended to limit the scope of the exemplary embodiments of the present disclosure.

One of the objects of the present disclosure is to provide a wearable microsystem/device to deliver a focused light into a body (e.g., a human body) with application(s) to photopharmaceuticals and optogenetics. The exemplary microsystem/device can also support a pulsed operation to further facilitate a two-photon excitation. In the exemplary context of delivering the light into the brain, applications can include spatially targeted delivery of anti-seizure, neuro-inhibitory pharmaceuticals for the treatment of epilepsy or the creation of a pharmacologic Wada test in which very localized anaesthesia can be employed.

Possible constraints of the exemplary microsystem/device can be associated with possible requirements that the power at the skin may not exceed the ANSI Z136.1-2022 maximum permissible exposure (MPE) limits [see, e.g., Ref. 1] as determined by heating. These same or similar exemplary heating constraints can be extended everywhere, e.g., in the brain, which facilitates an infrared neural stimulation, that is driven by heating effects, to be avoided [2]. For 850 nm, this exposure limit under continuous-wave (CW) conditions is given by 2000·^{100·002(λ-700)} W/m² for 1 between 700 and 1050 nm. This evaluates to 4000 W/m² or 4 mW/mm². If pulsed excitation is used, tolerable instantaneous power levels can be considerable higher as long as the pulse duty cycle is such that the average power of 4 mW/mm² is not exceeded over the time course of the pulse sequence. For a pulse width of τ=100 usec, the maximum tolerable instantaneous pulse is given by 1.1×10⁴·10^{0.002(λ-700)}τ^{0.25} J·m⁻² which is 22 W/mm².

According to the exemplary embodiments of the present disclosure, an exemplary wearable device for a focused direction of a light into a tissue and a method for providing such wearable device is provided. Such exemplary device can comprise an array of laser devices, and a configuration that can include metasurfaces and/or spatial light modulators (SLMs). The exemplary metasurfaces and/or the spatial light modulators can facilitate a wavefront shaping for each of the laser devices.

According to certain exemplary embodiments of the present disclosure, the laser devices can be or include solid-state lasers and/or vertical-cavity surface-emitting lasers. The configuration can include both the metasurfaces and the SLMs to generate one or more diffraction limits. The laser device can be provided as nodes, whereas each of the nodes can comprise chips of complementary metal-oxide-semiconductor (CMOS) integrated circuits. The laser devices can be mounted on the chips in each of the nodes. The chips can include the drive electronics for the laser devices. Alternatively or in addition, the chips can include an array of photodetectors configured to measure a return radiation that scatters back from the tissue after a delivery of the light into the tissue. The photodetectors can be or include single-photon avalanche diodes. At least one or all of the chips can contain all of measurement electronics which can be configured to perform a time-domain diffuse optical tomography procedure. The measurement electronics can include time-to-digital converters and digital control logic.

In yet further exemplary embodiments of the present disclosure, a flexible printed circuit board can be provided which can conform to a surface of a body that includes the tissue. The CMOS integrated circuits can be mounted on the flexible printed circuit board. The exemplary device can be wireless and/or battery-powered.

These and other objects, features, and advantages of the present disclosure will become apparent upon reading the following detailed description of the exemplary embodiments of the present disclosure, when taken in conjunction with the appended paragraphs.

### BRIEF SUMMARY OF THE INVENTION

The present disclosure provides a device for delivering near-infrared (NIR) light into the tissue of a subject, wherein the device comprises:
(a) one or more batteries;
(b) one or more Complementary Metal-Oxide-Semiconductor (CMOS) chiplets;
(c) one or more rigid electronics boards;
(d) one or more flexible polyimide boards;
(e) one or more laser devices;
(f) one or more metasurface chiplets; and
(g) one or more spatial light modulators (SLMs).

The present disclosure provides a method of activating one or more photoswitch molecules in a subject, wherein the subject wears a device for delivering near-infrared (NIR) light into the tissue of a subject, wherein the device comprises:
(a) one or more batteries;
(b) one or more Complementary Metal-Oxide-Semiconductor (CMOS) chiplets;
(c) one or more rigid electronics boards;
(d) one or more flexible polyimide boards;
(e) one or more laser devices;
(f) one or more metasurface chiplets; and
(g) one or more spatial light modulators (SLMs).

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is a side perspective cut-away view of at least one portion of the device, according to exemplary embodiments of the present disclosure.

FIG. 2 is an illustration of an electronic design that includes ASIC and an interface dongle, according to exemplary embodiments of the present disclosure.

FIG. 3 is an exemplary table of specifications for the exemplary device, according to various non-limiting exemplary embodiments of the present disclosure.

FIG. 4 is a set of illustrations of a full stack-up at one pixel in the patch, according to exemplary embodiments of the present disclosure.

FIG. 5 is a set of side-view illustrations of an exemplary mechanism using which exemplary metasurface and SLMs work together to focus light at depth in the tissue, according to exemplary embodiments of the present disclosure.

FIG. 6 is a set of side-view illustrations providing the exemplary interactions in the device so as to provide an importance of the metasurface to produce strong light focusing, according to exemplary embodiments of the present disclosure.

FIG. 7 is a top view illustration of an exemplary representation of a chiplet array on an 8×8 device, according to exemplary embodiments of the present disclosure.

FIG. 8 is a set of exemplary illustrations providing exemplary spacing and layout of the chiplets on the patch, according to exemplary embodiments of the present disclosure.

FIG. 9. Representative simulations showing focusing λ=850 nm light at 1.5-cm depth in a scattering tissue. (a) Schematic illustration showing that the NIR-HEAD patch combines the contributions from a large array of 128 VCSELs (i.e., 8×8 chiplets, each containing two VCSELs) to form a strong focal spot 1.5 cm deep in the tissue. (b) Full-wave computations of light intensity distributions over a monitoring area of 1 mm×34 mm that is 15 mm deep in a skull tissue. The light coupled into the tissue in this simulation is an array of 33×33 high-NA beams distributed over an area of 1 mm². The results show that (i) in the absence of phase modulation of the incident beams, light diffusively spreads over the monitoring plane (top subfigure in (b)); (ii) with proper phase modulations over the incident beams, light can be focused at any desired lateral distances from where it is injected into the tissue (bottom four subfigures in (b)). (c) Raster-scanning a tight focal spot over a large volume (total scanning time shorter than τₚₕₒₜₒ).

FIG. 10. Waveguide design choices for AWARE. (a) Original Phase 0 design. This design made of a Ø=3 μm lens has a moderate output divergence (NA-0.57) but has nearly impossible alignments specs of 10 nm in z and 50 nm in x and y. (b) Current Phase 0 design. These challenges were overcome by an alternate design in Phase 0, which allows for a larger lens and easier alignment. There is efficient coupling (>90%) to the waveguide yet the design delivers high output divergence (NA=0.9) and a point-source output that yields a constant phase front. The challenges with this design, however, is that the film oxidation between VCSEL transfer is difficult to fabricate and the interposer thickness is still large (600 μm). (c) Phase 1 design. Because of these challenges, the present disclosure discloses the same lens design but with a waveguide tree which allows the metasurface-SLM cap to be reduced to 40 μm, while providing uniform intensity and phase for the fields at the metasurface. The performance of the design is less sensitive to output NA with a simplified metasurface design. The film is oxidized after transfer to the metasurface to ease fabrication challenges.

FIGS. 11A-11B. SLM pixel design and metasurface design. (a) One pixel of the SLM with organic thin-film transistors. (b) Metasurface phase distribution for generating an array of 4×6 focal spots with a pitch of 7.5 μm. The focal spot array is superimposed over the liquid crystal (actively tunable) portion of the SLM pixel design in (a).

FIGS. 12A-12B. Inferring focal spot formation and location using back-scattered light. (a) Schematic illustration showing that the formation of a focal spot in the brain is associated with a number of back-scattered bright spots on the skin. The lateral extent over which the latter spread is approximately twice the lateral location of the focal spot. (b) Full-wave computations showing two examples of the concurrent formation of the focal spot in the scattering tissue (top subfigures) and the bright reflected spots on the skin surface (bottom subfigures). Importantly, the reflected features are not observed in the absence of focusing.

FIGS. 13A-13B. NIR-HEAD system. (a) 8×8 array of chiplets with two VCSELs per chiplet. (b) TD-DOT measurement capabilities on the patch. Average histogram behavior can be used to extract absorption and scattering coefficients as a function of position. “Fine-structure” can be used to calibrate focusing.

FIG. 14. Assembly of the NIR-HEAD patch. Key components include the LCD transmission SLM, the lens-waveguide-metasurface chiplets, the CMOS chiplets, the VCSELs, the flexible polyimide package, the rigid electronics board, and the LiPo battery.

Throughout the drawings, the same reference numerals, and characters, unless otherwise stated, are used to denote like features, elements, components, or portions of the illustrated embodiments. Moreover, while the present disclosure will now be described in detail with reference to the figures, it is done so in connection with the illustrative embodiments and is not limited by the particular embodiments illustrated in the figures and the appended paragraphs.

### DETAILED DESCRIPTION OF THE INVENTION

The present disclosure provides a device for delivering near-infrared (NIR) light into the tissue of a subject, wherein the device comprises:
(a) one or more batteries;
(b) one or more Complementary Metal-Oxide-Semiconductor (CMOS) chiplets;
(c) one or more rigid electronics boards;
(d) one or more flexible polyimide boards;
(e) one or more laser devices;
(f) one or more metasurface chiplets; and
(g) one or more spatial light modulators (SLMs).

In some embodiments, the device has the ability to focus the NIR light into the tissue of the subject.

In some embodiments, the distance between the focal point of the focused NIR light and the tissue is from 1 mm to 50 mm.

In some embodiments, the distance between the focal point of the focused NIR light and the tissue is from 10 mm to 50 mm.

In some embodiments, the distance between the focal point of the focused NIR light and the tissue is from 10 mm to 40 mm.

In some embodiments, the distance between the focal point of the focused NIR light and the tissue is from 15 mm to 40 mm.

In some embodiments, the distance between the focal point of the focused NIR light and the tissue is from 15 mm to 30 mm.

In some embodiments, the device has the ability to detect backscattered photons.

In some embodiments, the battery is lithium battery.

In some embodiments, the battery is lithium polymer (LiPO) battery.

In some embodiments, the device comprises 8×8 CMOS chiplets.

In some embodiments, each chiplet comprises two laser devices.

In some embodiments, the CMOS chiplets contain software drivers.

In some embodiments, the CMOS chiplets comprise single-photon avalanche diodes (SPADs).

In some embodiments, each CMOS chiplet comprises an array of 20×20 SPADs.

In some embodiments, the array of 20×20 SPADs measures the time-of-flight (ToF) of backscattered photons.

In some embodiments, the CMOS chiplets are mounted to the flexible polyimide board.

In some embodiments, the rigid electronics board comprises components of signal processing, digital control, power management, battery charging, decoupling capacitance, and wireless interface.

In some embodiments, the rigid electronics boards comprise Field-Programmable Gate Arrays (FPGAs) and a platform comprising a processor and a memory.

In some embodiments, the rigid electronics board is a printed-circuit board (PCB).

In some embodiments, the rigid electronic board connects to the flexible polyimide board and connects to the SLMs.

In some embodiments, one or more zero-insertion-force (ZIF) connectors connect the rigid electronics board to the SLMs.

In some embodiments, the flexible polyimide board is flexible PCB.

In some embodiments, the CMOS chiplets are mounted to the flexible PCB.

In some embodiments, the laser devices are vertical cavity surface emitting lasers (VCSELs).

In some embodiments, VCSELs employ AlₓGa₁₋ₓAs/Al_{y}Ga_{1-y}As distributed Bragg reflectors.

In some embodiments, VCSELs comprise an active region comprising multiple quantum wells of In_{y}Ga_{1-y}As/Al₁₋ₓGaₓAs or InGaAs/GaAs₁₋ₓPₓ. VCSELs.

In some embodiments, each VCSEL has an output power from 1 to 20 mW.

In some embodiments, each VCSEL has an output power from 1 to 10m mW.

In some embodiments, each VCSEL has an output power of 5 mW.

In some embodiments, each VCSEL emits a laser at a wavelength from 600 to 900 nm.

In some embodiments, each VCSEL emits a laser at a wavelength from 650 to 850 nm.

In some embodiments, each VCSEL emits a laser at a wavelength at 650 nm, 700 nm, 750 nm, 800 nm, or 850 nm.

In some embodiments, each of the metasurface chiplet further comprises
(a) a lens;
(b) a network of optical waveguides (waveguide tree); and
(c) metalenses.

In some embodiments, when an input laser pass through the lens, the waveguide tree evenly splits the laser into multiple output emissions over an area.

In some embodiments, the waveguide tree evenly splits the laser into 16×16 output emissions over an area of 1 mm×1 mm.

In some embodiments, metalenses then convert the 16×16 output emissions into 64×96 high Numerical Aperture (NA) focal spots as inputs for the SLM.

In some embodiments, the metasurface comprises 2D arrays of silicon nano-pillars.

In some embodiments, the nano-pillars have a height of 700 nm and diameters from 100 nm to 290 nm.

In some embodiments, the SLMs shape the wavefront of emission from each of the VCSELs.

In some embodiments, the SLMs produce a phase shift of up to about 0.67π or about 120° for the 850-nm light at the normal incident angle.

In some embodiments, the SLM comprises a liquid crystal layer (LCD) encapsulated by flexible and ultra-thin membranes.

In some embodiments, the device delivers a power of at least 4 mW per mm² of the subject's tissue.

In some embodiments, the subject is a mammal.

In some embodiments, the mammal is a human.

In some embodiments, the tissue is brain tissue.

In some embodiments, the device delivers near-infrared (NIR) light into about 3 cm below the surface of the skull of the subject.

The present disclosure provides a method of activating one or more photoswitch molecules in a subject, wherein the method comprises the subject wearing a device for delivering near-infrared (NIR) light into the tissue of the subject, wherein the device comprises:
(a) one or more batteries;
(b) one or more Complementary Metal-Oxide-Semiconductor (CMOS) chiplets;
(c) one or more rigid electronics boards;
(d) one or more flexible polyimide boards;
(e) one or more laser devices;
(f) one or more metasurface chiplets; and
(g) one or more spatial light modulators (SLMs).

In some embodiments, the one or more photoswtich molecule is photo-pharmaceuticals.

In some embodiments, the photo-pharmaceuticals are photoactivatable dextroamphetamines

The present disclosure provides a method of delivering focused light into a predetermined area of a tissue in a subject, wherein the method comprises the subject wearing a device, wherein the device comprises:
(a) one or more batteries;
(b) one or more Complementary Metal-Oxide-Semiconductor (CMOS) chiplets;
(c) one or more rigid electronics boards;
(d) one or more flexible polyimide boards;
(e) one or more laser devices;
(f) one or more metasurface chiplets; and
(g) one or more spatial light modulators (SLMs).

In some embodiments, the method further comprises administering the photoswitch molecules to the subject.

The present disclosure provides a method of treating a disease in a subject in a subject, wherein the method comprises the subject wearing a device, wherein the device comprises:
(a) one or more batteries;
(b) one or more Complementary Metal-Oxide-Semiconductor (CMOS) chiplets;
(c) one or more rigid electronics boards;
(d) one or more flexible polyimide boards;
(e) one or more laser devices;
(f) one or more metasurface chiplets; and
(g) one or more spatial light modulators (SLMs).

In some embodiments, the disease is epilepsy.

The present disclosure provides a method of controlling the activity of neurons in one or more target cells in a subject, wherein the method comprises the subject wearing a device, wherein the device comprises:
(a) one or more batteries;
(b) one or more Complementary Metal-Oxide-Semiconductor (CMOS) chiplets;
(c) one or more rigid electronics boards;
(d) one or more flexible polyimide boards;
(e) one or more laser devices;
(f) one or more metasurface chiplets; and
(g) one or more spatial light modulators (SLMs).

### Definitions

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. In embodiments, about means within a standard deviation using measurements generally acceptable in the art. In embodiments, about means a range extending to +/−10% of the specified value. In embodiments, about includes the specified value. Unless otherwise indicated, the word “or” in the specification and claims is considered to be the inclusive “or” rather than the exclusive or, and indicates at least one of and any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

As used herein, “photoswitch molecule” is a compound that reversibly changes its shape (isomerizes) and properties (like color, polarity, or fluorescence) when exposed to light, switching between two forms (isomers) that can also interconvert via heat or different light wavelengths, making them useful for controlling materials and biological systems with light.

As used herein, “a focal point (or focus)” in optics is the precise spot where parallel light rays converge (meet) after passing through a converging lens or reflecting off a concave mirror, forming a sharp image, or where they appear to spread from in a diverging lens/convex mirror, crucial for imaging in cameras, eyes, telescopes, and microscopes.

As used herein, “Near-infrared (NIR) light” is a part of the electromagnetic spectrum with longer wavelengths than visible red light (roughly 700-2500 nm), invisible to the human eye.

As used herein, “backscattered photons” are photons (light or radiation particles) that have been scattered backward, away from their original direction, after interacting with matter, often returning toward the source.

As used herein, “Time of Flight (ToF)” refers to the duration an object, signal, or particle takes to travel a certain distance.

As used herein, a “Field-Programmable Gate Array (FPGA)” is a reprogrammable silicon chip containing configurable logic blocks (like LUTs) and programmable interconnects, allowing users to define its function after manufacturing for tasks like ASIC prototyping, high-speed processing, and custom hardware acceleration, offering flexibility unmatched by fixed-function chips.

As used herein, a “Bragg reflector (or Distributed Bragg Reflector, DBR)” is a mirror made of many thin, alternating layers of materials with high and low refractive indices, designed to strongly reflect light within a specific wavelength range (a “stopband”) through constructive interference, acting as a photonic crystal for high-quality, low-loss reflection crucial in lasers, optical filters, and fiber optics.

As used herein, a “metalens (metasurface lens)” is a flat, ultrathin optical lens that manipulates light using engineered nanoscale structures (meta-atoms) instead of traditional curved surfaces, allowing for precise control over light's phase, amplitude, and polarization to focus, shape, and steer beams, offering advantages in miniaturization, weight, and potentially cost for applications in cameras, medical imaging, and sensing.

As used herein, a “Vertical Cavity Surface Emitting Laser (VCSEL)” is a semiconductor laser that emits light perpendicular (vertically) to the wafer surface, featuring a short, vertically aligned cavity with highly reflective mirrors (Distributed Bragg Reflectors or DBRs) sandwiching a thin active region, allowing for compact size, high-speed operation, low power, and easy integration into 2D arrays.

As used herein, an “optical waveguide” is a structure, like an optical fiber or a path on a photonic chip, that guides light (electromagnetic waves) over distances with minimal loss, using materials with different refractive indices to confine light via total internal reflection (TIR).

As used herein, a “high Numerical Aperture (NA)” means an optical system (like a microscope objective or camera lens) can collect more light and resolve finer details, leading to sharper, brighter, and higher-resolution images.

As used herein, a “Spatial Light Modulator (SLM)” is an optical device that electronically controls the spatial pattern of light's intensity, phase, or polarization, acting like a programmable hologram or variable diffraction grating to shape light beams for applications in holography, laser beam shaping, microscopy, AR/VR, and optical communication. It typically uses liquid crystals on a microdisplay to dynamically alter the wavefront of light in real-time, enabling complex optical functions.

As used herein, a “metasurface” is an engineered, ultrathin, artificial material that can manipulate electromagnetic waves like light, sound, or microwaves by controlling their phase, amplitude, and/or polarization. These surfaces can be made of a carefully arranged pattern of subwavelength elements called “meta-atoms”.

As used herein, “photo-pharmaceuticals” (or photopharmacology) regards drugs modified with light-sensitive “photoswitches” to control their activity with light, allowing for precise activation, turning them on or off with specific light wavelengths to achive therapeutic or prophylactic effect, e.g, target diseases like cancer, infections, or mental health issues, and minimizing side effects by avoiding surrounding tissue damage.

### General

For the foregoing embodiments, each embodiment disclosed herein is contemplated as being applicable to each of the other disclosed embodiments.

As used herein, all headings are simply for organization and are not intended to limit the disclosure in any manner. The content of any individual section may be equally applicable to all sections. All combinations of the various elements disclosed herein are within the scope of the invention.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

The following is intended to be a description of the exemplary embodiments of the present disclosure, and is not intended to limit the scope of the exemplary embodiments of the present disclosure.

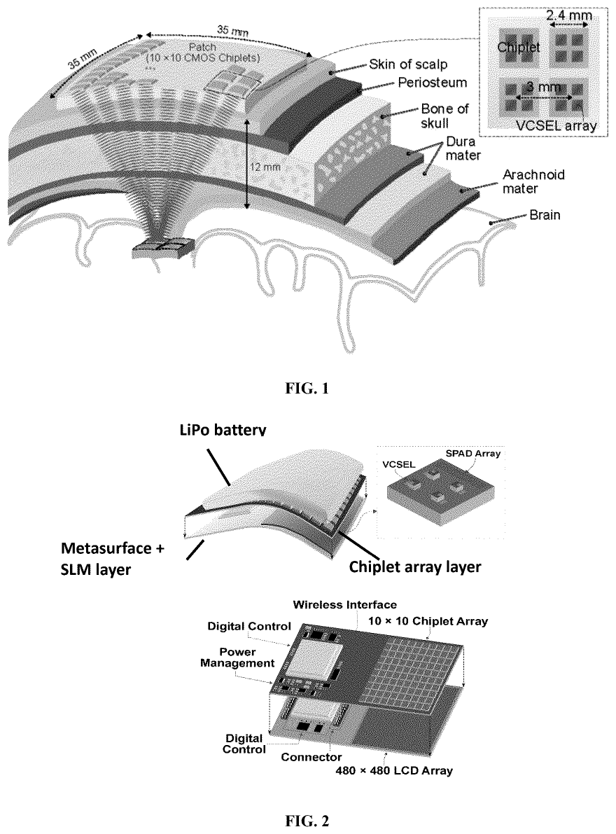

Scattering (primarily Rayleigh scattering with 1/λ⁴ wavelength dependence) along with absorption can be the principal impediment to getting light to depths exceeding 15 mm into tissue and controlling its spatial extent. For light into the head, the mean-free path (2 mfp) is about 1.4 mm in the scalp, leading to almost completely diffusive transport by 4 mm into the scalp. Wavefront shaping can overcome such scattering, which can be realized with metasurfaces and spatial light modulators (SLMs) placed over each VCSEL site, taking advantage of the coherence that exists over the surface of the VCSELs. According to the exemplary embodiments of the present disclosure, these metasurfaces and SLMs can shape the wavefronts [see, e.g., Ref. 3] to produce as close to a collimated beam as possible at the target location at depth (see FIG. 1).

The exemplary compensation for individual variation in the wavefront shaping can be accomplished by the addition and/or use of the SLMs. Even with these wavefront-shaped sources, one must overcome the absorption that will consume heavily scattered photons. This can be performed by maximizing the number of wavefront-corrected coherent sources that can be directed at the target; these sources are otherwise incoherent with respect to each other. For example, all sources can be directed to the target location with time-domain diffuse optical tomography (TD-DOT) measurement capability in the device used to tune the SLMs on the patch to achieve both the desired power density (e.g., within safety limits) and desired stimulation volume. In a pulsed operation, the emitted beams of more VCSEL sources can be potentially superimposed because of the higher safety limits. To provide an opportunity for a maximum adaptability and a maximum power delivery, the largest scalp surface area possible should be illuminated with the maximum allowable average optical power density of 4 mW/mm²

According to the exemplary embodiments of the present disclosure, it is possible to select a target surface area of 1000 mm², which can allow up to 4 W to be delivered at the surface. In one exemplary non-limiting embodiment, not all of the surface area of the scalp can be illuminated by pixels because some area can be needed for interconnect and electronics. This exemplary implementation can amount to a fill-factor efficiency of approximately η_{FF}=0.44. This reduction can mean that the total average power that can be delivered at the 1000 mm² surface is reduced to about 1.76 W.

These exemplary requirements can drive the entire patch geometry of the exemplary embodiment of the exemplary device as shown in FIG. 1. Such an exemplary device illustrated in FIG. 1 can have, e.g., a 35 mm×60 mm flexible patch design that contains, on a 35 mm×35 mm portion of the patch, a 10×10 array of about 2.4 mm×2.4 mm complementary metal-oxide-semiconductor (CMOS) chiplets on a 3-mm pitch. Each chiplet contains four independently controllable about 0.22 mm×0.22 mm, multi-mode vertical-cavity surface emitting lasers (VCSEL). These VCSELs, which can be custom fabricated depending on wavelength requirements, can be similar to commercially available 850-nm devices that can deliver 5 mW continuous-wave (CW) emission at a wall-plug efficiency of 30%. Spectral 20 bandwidths for these exemplary designs can be 2 nm (FWHM). Metasurfaces and SLMs can allow the power from these devices to be directed into about a 1 mm² collimated beam for each chiplet at a given angle into the head, compensating for scattering with wavefront shaping based on calibration. In an exemplary CW operation, the exemplary 4 mW/mm² constraint can limit the VCSEL array to, e.g., 4 mW operation. The exemplary electronics can also support pulse operation with pulse widths as short as about 2 nsec.

Beyond being a light source, the patch can verify the device coupling to the scalp and to measure the scattering and absorption properties of the tissue once the patch is positioned as part of a system calibration. This can be done by using, e.g., TD-DOT, building on capabilities that have already been developed for brain imaging [see, e.g., Refs. 4 and 5]. TD-DOT can allow for an evaluation of the quality of the metasurfaces for the particular individual and device placement. In particular, the efficacy of wavefront correction attained by the metasurfaces for light transmitting through the scattering tissue can be quantified by a reduction of the distribution of time-of-flight (DTOF) of photons. The SLMs can then be adjusted to improve the focusing. To support this TD-DOT capability, the chiplets all have TD-DOT measurement capability. Each chiplet contains an array of single-photon avalanche diodes (SPADs) capable of detecting photons scattered back as well as the arrival time of these photons for this purpose. For example, a small additional FF reduction can be used to achieve this.

For an exemplary worst-case total average optical output power for the array of about 1.76 W, this can provide an input electrical power of about 5.9 W from the wall-plug efficiency. Adding in the addition power overhead associated with the efficiency of the laser drivers, the predriver control, the TD-DOT functionality on some of the chiplets, and the wireless 802.11n interface to the device, the estimated total average power of the device is 6.5 W. For an exemplary goal of about one hour of continuous operation, e.g., 6.5 W-h of battery can be used. In one example, lithium polymer (LiPo) batteries of a specific energy up to 265 W-h/kg can provide 6.5 W-h of storage in less than 24 g.

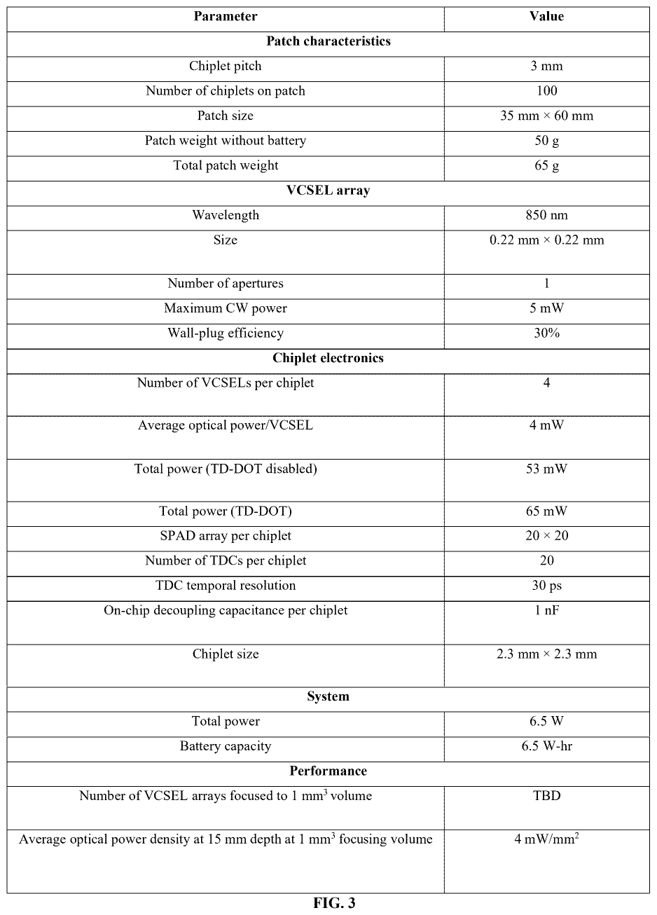

According to an exemplary embodiment of the present disclosure, digital control, power management, battery charging, decoupling capacitance, and the wireless interface can be included on the 35 mm×25 mm portion of the flexible PCB outside of the array (as shown in FIG. 2), which incorporates a stiffener for more robustness. For example, at approximately 750 W-h/L, a 6.5 W-h battery should used a volume of approximately 8.6 mL. If the entire area of the patch is used for such mechanically flexible LiPo battery, this can amount to, e.g., 1 mL at a 5-mm thickness, which is smaller than the required volume. In FIG. 2, the battery is illustrated in the form factor of the patch, although larger batteries can be required and/or utilized, extending the size of the patch, particularly to meet goals of longer continuous operation. FIG. 3 shows additional details of the exemplary specification of such exemplary embodiment of the design.

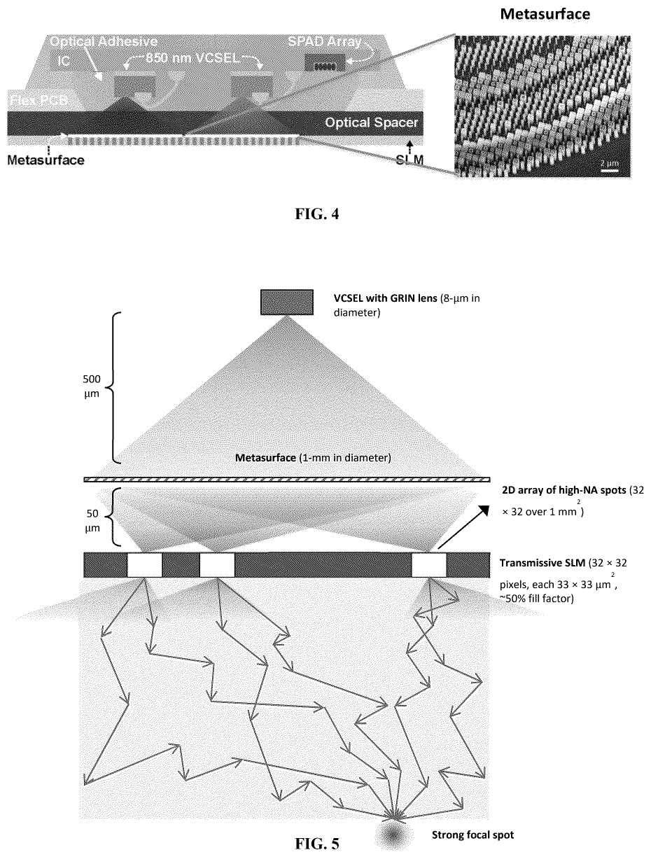

Components of the system include:
Exemplary CMOS chip-stacked design for VCSELs and electronics, flexible printed-circuit board design. This can be similar to the devices focused on wearable device for NIR brain imaging [see, e.g., Refs. 4 and 5]. For example, VCSEL can be attached to the driving die with silver epoxy used to make the cathode connection and the anode wirebonded with low-profile wirebonds with a loop height of less than 100 μm. Chiplets can be bonded into a polyimide flexible printed-circuit board (PCB) using anisotropic conductive films (ACF) or solder-ball attached. The entire exemplary stack-up according to the exemplary embodiments of the present disclosure is shown in FIG. 4, including exemplary metasurfaces and SLMs described herein.
Exemplary Electronics to support high-power pulsed mode operation. While still supporting CW operation, the exemplary devices can support the requirements dictated by the two-photon (2p). For example, the number of 2p excitations per molecule is given by

$$N = \frac{1}{2} {σ}_{2} {I}^{2} τ ( \frac{{t}_{stim}}{{t}_{period}} ) ,$$

where σ₂ is the 2p cross section, τ is the pulse width, tₛₜᵢₘ is the total stimulation time, and t_{period} is the pulse period. Because of the I² dependence of this activation, pulsing can increases this activation by a factor of t_{period}//tₛₜᵢₘ, which can be the same as the ratio of the pulse power to the average power. The nonlinearity of 2p operation also helps to localize the area of activation. The principal challenge of 2p operation can be overcoming the low 2p cross sections. Pulse width and period can be partially or completely programmable such that these may be adjusted depending on the requirements for the therapeutic. It is also possible to increase laser power beyond the limits noted above if we can demonstrate device safety.
Exemplary Metasurfaces. Exemplary dielectric metasurfaces can operate as highly-transparent phase masks with subwavelength resolution, e.g., to convert the multimode VCSEL emission into a fundamental Gaussian beam, steer this beam towards a 1-mm² target area at depth greater than 15 mm and compensate for the spatial distribution of accumulated wavefront error over the beam cross-section as the light wave propagate through the several layers of the phantom. Unlike conventional SLMs or diffractive optical elements (DOEs), which have a pitch several times larger than the operating wavelength, the subwavelength pitch and subwavelength meta-units used in metasurfaces can facilitate or ensure that the incident optical power is fully utilized to produce a single beam with a desired optical wavefront. These metasurfaces can be augmented by liquid-crystal-based SLMs which provide dynamic programmability of the wave-front shaping.
Exemplary Wireless interface. In one example, a wireless interface can be used to download the TD-DOT data for device calibration and to produce the appropriate phase configurations for the PDM interference. This wireless interface can rely on, e.g., the Broadcom BCM2711, Quad core Cortex-A72 (ARM v8) 64-bit SoC and the Infineon AIROCTM CYW43455 WiFi chip, which can be the same processor and WiFi chip used in the Raspberry Pi 4. The CYW43455 can support 5-GHZ WiFi 5 (802.11ac), which deliver a data bandwidth of 200 Mbps. It is possible—in one example—to utilize the software infrastructure (at least partially) from the Raspberry Pi for this interface. The BCM2711 interfaces to a Xilinx FPGA which can be used to control the patch and format the data for transfer between the patch and the BCM2711.
Exemplary TD-DOT measurements and calibration. The exemplary device according to the exemplary embodiment of the present disclosure can perform TD-DOT imaging after initial placement on the head, following recent work by us on patch-based imagers [see, e.g., Refs. 4, 5 and 7]. These exemplary TD-DOT measurements can be made with the same or similar metasurfaces and SLMs used for the light shaping such that the reconstructions and calibrations can consider these optics.

### Exemplary VCSEL Design

One exemplary embodiment of the exemplary device can be set for a nominal 850-nm wavelength. Further, according to other exemplary embodiments, other exemplary VCSELs exemplary VCSELs can be grown by metal organic chemical vapor deposition (MOCVD). Some or all of such exemplary structures can employ AlₓGa₁₋ₓAs/GaAs distributed Bragg reflectors for top and bottom mirrors with an active region can be used that include, e.g., multiple quantum wells of GaAs/Al₁₋ₓGaₓAs or (for longer wavelengths) In_{y}Ga_{1-y}As/GaAs₁₋ₓPₓ. These longer wavelengths have become popular for telecommunications applications due to their compatibility with optical fibers. The multimode VCSELs employed according to the exemplary embodiments of the present disclosure can have a FWMH beam divergence of 20°, which can be expanded as needed to illuminate the metasurface in the stack-up shown in FIG. 4, e.g., with the addition of a lens at its aperture.

Exemplary Metasurfaces and SLMs for Beamforming and to Compensate for Scattering with Wavefront Shaping.

It is known that under correct conditions, it is possible to take advantage of the linearity of scattering processes to compensate for scattering in turbid media through wavefront shaping [see, e.g., Refs. 3 and 12]. An exemplary metasurface can convert an output of each VCSEL into a 2D array of high numerical-aperture (NA) optical spots to fully access open eigenchannels in the scattering tissue. In one example, a transmissive SLM can be used to modulate the phase of each optical spot independently to ensure constructive interference between the open eigenchannels for maximal optical power delivery to a target spot in the scattering tissue.

According to an exemplary embodiment of the present disclosure, a scattering medium can contain a number of open eigenchannels with a high transmission coefficient. For example, each eigenchannel can correspond to a specific linear combination of multiple incident free-space waves. These exemplary eigenchannels should be excited to synthesize a strong focal spot inside the medium. The chance of accessing these open eigenchannels increases significantly when waves over a large angular range (i.e., high numerical aperture or NA) are incident at the scattering medium:

$${N}_{open\_channel} ∝ \frac{A ( NA ) 2}{λ2}$$

The exemplary metasurface and SLM in front of each of the 20×20 VCSELs can be designed to (i) convert the multimode VCSEL emission into a fundamental Gaussian beam, (ii) steer such beam towards a 1-mm² target area at a depth of 15 mm in the phantom, and (iii) compensate for the spatial distribution of accumulated wavefront error over the beam cross-section as the light wave propagate through the several layers of the phantom. The exemplary phase profile for achieving the capabilities (i) and (ii) described above can be determined and/or established by, e.g., the angular distance between the target area and the surface normal of the VCSEL, as well as the phase profile of the initial multimode VCSEL laser beam. The exemplary metasurfaces can include 2D arrays of silicon nitride nano-pillars with a pitch of approximately half of the wavelength (425 nm). The exemplary diameter of the nano-pillars can be selected to change the local effective refractive indices so that collectively an array of, e.g., ˜2350×2350 nano-pillars over an area of about 1 mm² can finely sample and implement the phase profile determined by the optimization process based on the SLM. An example metasurface according to the exemplary embodiment of the present disclosure is shown in FIG. 4.

FIG. 5 shoes an exemplary mechanism by which metasurface and SLM work together to focus light at depth in the tissue, and illustrates shows how light is focussed with the above described exemplary design combination of exemplary metasurfaces and SLMs. For example, the metasurface can convert VCSEL emission into a 2D array of diffraction-limited spots, each with large emission angles (NA˜0.9), to fully access open eigenchannels. There can be a high transmission of the 2D spot array through actively tuneable portions of the transmissive SLM, ensuring constructive interference between all open eigenchannels Incident light waves with high angular range and tuneable phase lead to a strong focal spot at depth in the scattering tissue in the presence of stationary and dynamic individual variations.

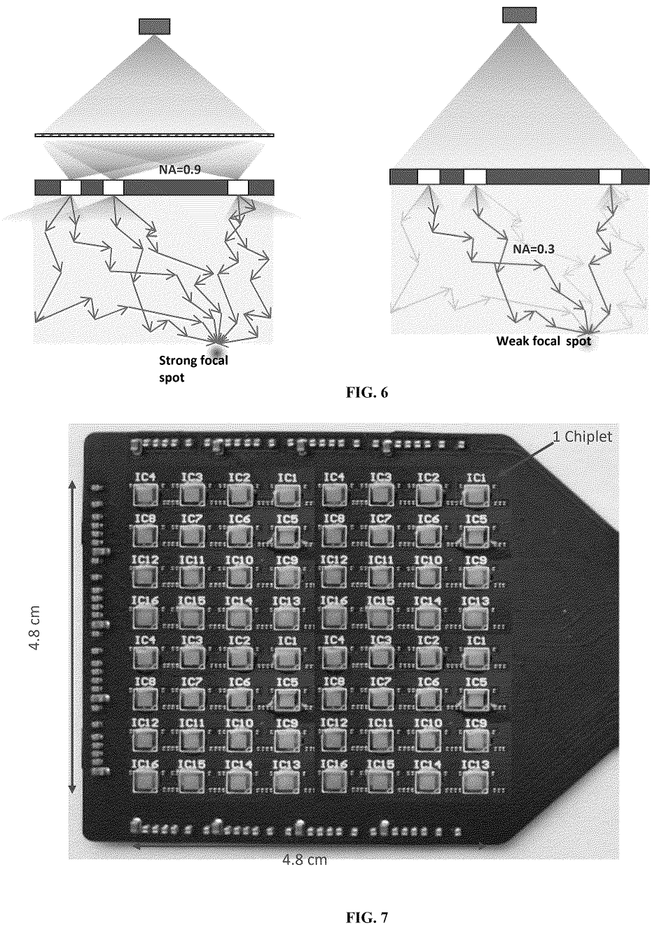

To achieve a reconfigurability to maximize focal spot power and conform to individual variation, it is possible to combine, at each VCSEL site, e.g., a 1-mm-by-1-mm metasurface with a transmissive LCD SLM (32×32 pixels with 33-mm pitch) to reconfigurably tune the relative phase of 32×32 high-NA optical spots injected into the scalp. Without the metasurface, there could be a reduced transmission through SLM and an incomplete access of open eigenchannels, as shown in FIG. 6.

### Exemplary Chiplets for VCSEL Drive Electronics

Exemplary CMOS chiplets containing the laser drivers and predrivers can be fabricated in a CMOS technology, which can also incorporate the TD-DOT measurement capabilities. Because of the high current transient demands of a pulsed operation, it can be important to minimize or otherwise reduce the inductance of the connection between the driver and the VCSELs, which can be accomplished in major part by, e.g., the stacking of the VCSELs on the CMOS chiplet containing both the driver and pre-drivers and using several wirebonds for the top anode connection. It is possible to reach a preferred driver performance using, e.g., exemplary 2.5-V CMOS devices in the CMOS technology, which follows approaches provided for a wearable TD-DOT imaging device [see, e.g., Refs. 4 and 5]. Significant on-chip decoupling capacitance can also be provided on the chiplets (see FIG. 1), e.g., in the pulse mode operation.

### Exemplary CMOS Chiplets for TD-DOT Measurement Capabilities, Reconstruction, and Calibration

This exemplary capability can be based on prior designs [see, e.g., Refs. 4, 5 and 7], e.g., a flexible patch for NIR imaging of hemodynamics in the brain, which has a similar form factor to the device. Each of the exemplary chiplets can provide a TD-DOT measurement functionality. Importantly for this exemplary application, data collected from these TD-DOT measurements can be used to calibrate the SLMs to maximize the effectiveness of focussing. In addition, this exemplary measurement capability ensure that most or all the lasers of the exemplary device are properly coupled to the scalp. One of the differences in the exemplary system according to the exemplary embodiments of the present disclosure over generic TD-DOT measurement systems can be that the pulsed optical excitation is passing through the metasurfaces and SLM. The modelling can be utilized to incorporate this wavefront shaping.

In this exemplary case, the lasers can be pulsed chiplet-by-chiplet with a pulse width of only 2 nsec at a peak power on the scale of 50-100 mW at repetition rates as high as 50 MHz. For all the chiplets that are provided for the measurement, the SPAD arrays can simultaneously measure the time-of-flight of detected photons. The resulting arrival-time histograms provide information about the scattering and absorption of the tissue in the optical path. By measuring and collecting histograms at each site, an exemplary tomographic model can be provided using, e.g., inverse imaging procedures. Most or all of the TD-DOT measurements can be synchronized and triggered together through a shared external reference clock. The 20×20 SPAD array can have 20 dedicated event-driven time-to-digital converters, which can support exemplary detection and time-of-flight measurements. SPADs can operate by in-pixel quench-and-reset circuits.

For example, as previously described in Ref. 17, PHOEBE can be used to process the data from the TD-DOT measurement hardware to calculate the scalp coupling index (SCI). For each source location, it is possible to calculate the SCI to all of the detectors. The photon count as a function of time trace can be filtered to the frequency band from about 0.5 Hz to 2.5 Hz that includes the heart rate component at around 1 Hz. After normalizing each channel to its standard deviation, it is possible to determine or compute the cross-correlations. Exemplary correlations close to one indicate good source (and detector) coupling. This can be used to debug situations in which one or more optical sources are not in good contact with the skin, causing inefficient coupling of light into the scalp.

When the NIR-HEAD patch is originally placed on a subject, it can perform one or more TD-DOT measurement. For example, the exemplary measurement data can be obtained from the patch wirelessly, and image reconstructions can be performed in the cloud. The exemplary results of this analysis can be used to predict the efficacy of the metasurfaces and SLM to focus the light. Exemplary control of the exemplary device and reading of the exemplary measurement data can be done via, e.g., a wireless interface.

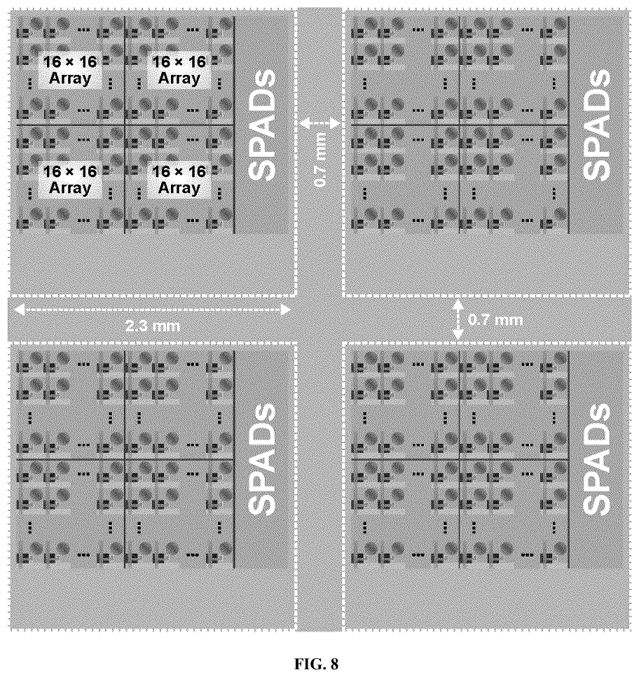

An exemplary configuration of the placement/mounting of the chiplets on the flexible patch is shown in FIG. 7. The exemplary general structure of the exemplary patch is shown in FIG. 8, e.g., for an exemplary 2×2 chiplet section of the array. The exemplary metasurfaces can present 256 transverse mode, each of which can pass through one pixel of the SLM. The SPAD arrays can be unblocked by the metasurfaces and SLM pixels, and can be set for no phase shift over the SPAD array. For example, 256 diffraction-limited spots can be create by the metasurface acting on the coherent light from a single VCSEL.

In this description, numerous specific details have been set forth. It is to be understood, however, that implementations of the disclosed technology can be practiced without these specific details. In other instances, well-known methods, structures, and techniques have not been shown in detail in order not to obscure an understanding of this description. References to “some examples,” “other examples,” “one example,” “an example,” “various examples,” “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described may include a particular feature, structure, or characteristic, but not every implementation necessarily includes the particular feature, structure, or characteristic. Further, repeated use of the phrases “in one example,” “in one exemplary embodiment,” or “in one implementation” does not necessarily refer to the same example, exemplary embodiment, or implementation, although it may.

As used herein, unless otherwise specified the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

While certain implementations of the disclosed technology have been described in connection with what is presently considered to be the most practical and various implementations, it is to be understood that the disclosed technology is not to be limited to the disclosed implementations, but on the contrary, is intended to cover various modifications and equivalent arrangements included within the scope of the appended numbered paragraphs. Although specific terms are employed herein, they are used in a generic and descriptive sense only and not for purposes of limitation.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification and drawings, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

Throughout the disclosure, the following terms take at least the meanings explicitly associated herein, unless the context clearly dictates otherwise. The term “or” is intended to mean an inclusive “or.” Further, the terms “a,” “an,” and “the” are intended to mean one or more unless specified otherwise or clear from the context to be directed to a singular form.

This written description uses examples to disclose certain implementations of the disclosed technology, including the best mode, and also to enable any person skilled in the art to practice certain implementations of the disclosed technology, including making and using any devices or systems and performing any incorporated methods. The patentable scope of certain implementations of the disclosed technology is defined in the numbered paragraphs, and may include other examples that occur to those skilled in the art. Such other examples are intended to be within the scope of the numbered paragraphs if they have structural elements that do not differ from the literal language of the numbered paragraphs, or if they include equivalent structural elements with insubstantial differences from the literal language of the numbered paragraphs.

### Example 1

### Considerations:

Effectiveness of focusing based on “fine-structure” calibration to compensate for scattering over thick tissue layers and reach resolution targets of the BAA
Ability to deliver adequate optical power at depth for the target therapeutic.
Packaging complexity, particularly alignment.
Heatsinking to manage power levels for patch of up to 6.5 W

### Approaches:

Patch illumination device containing a 8×8 array of CMOS chiplets, each containing two vertical-cavity surface-emitting lasers (VCSELs)
Each VCSEL can generate 5 mW over a 1 mm² scalp area.
Each light emitter utilizes a waveguide tree, metasurface, and programmable transmissive spatial light modulator to compensate for scattering and focus the light into the brain.
VCSELs are mounted on CMOS chiplets for drive and control.
CMOS chiplets implement time-domain diffuse optical tomography (TD-DOT) which can be used to measure coupling of patch to scalp and characterize absorption and scattering coefficients as well as measure “fine structure” in the histogram responses which can be used to control SLM focusing.
Modeling from measurement data allows combination of emitters to be chosen to meet stimulation requirements (volume, power)
Wireless interface for retrieve measurement data and configure emitters.
Thin flexible LiPo battery for powering.

### Phases Section

The present disclosure comprises multiple embodiment phases, such as phases 0, 1, and 2.

### 4.2. VCSEL Design

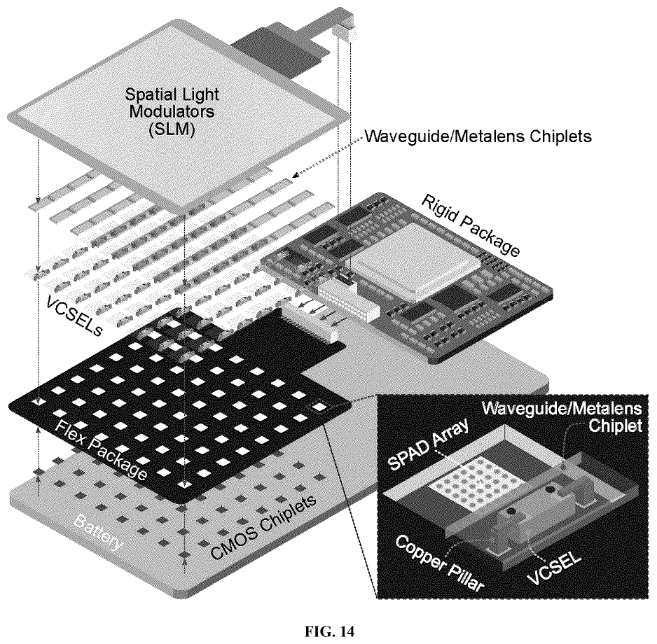

Custom VCSELs will be designed at the specific wavelength as required by the therapeutic, which will be in the wavelength range from 650 to 850 nm. These will take the form of two-VCSEL chiplets bonded to the surface of the CMOS chiplets as shown in FIG. 14. These VCSELs 8-10 are all grown by metal organic chemical vapor deposition (MOCVD); in Phase 1, the present disclosure will move to larger-scale manufacturing of these VCSELs. In some exemplary embodiments, the microlasers all employ AlₓGa₁₋ₓAs/Al_{y}Ga_{1-y}As distributed Bragg reflectors for top and bottom mirrors with an active region that consists of multiple quantum wells of In_{y}Ga_{1-y}As/Al₁₋ₓGaₓAs or (for shorter wavelengths) InGaAs/GaAs₁₋ₓPₓ. VCSELs with wavelengths longer than 850 nm VCSELs are predominantly manufactured today for sensing applications¹¹ and thus the VCSELs in this program will require custom epitaxial materials. Below summarizes the VCSEL design considerations.

### VCSEL Design for the NIR-HEAD Device

### Phase 0 Effort

In Phase 0, the present disclosure focused on the development of a custom single-mode 850-nm VCSELs through control of the oxide aperture diameter using off-the-shelf epitaxial VCSEL wafers. Single-mode operation is essential to avoid producing optical modes that are not efficiently coupled into the waveguide trees, and can also drift as a function of bias and temperature.

### High-Power Single-Mode VCSEL Designs

In Phase 1, the present disclosure will manufacture custom two-VCSEL chiplets for the five specific wavelengths using conventional single junction epitaxy and oxide aperture control, where the 5-mW objective can be straightforwardly achieved using custom conventional epitaxy for VCSEL wavelengths greater than 700 nm. However, achieving 5-mW Gaussian-mode output at 700 nm and especially at 650 nm wavelength will require the use of dual-junction cascaded VCSEL active regions to increase the VCSEL quantum efficiency above 100%. This will be accomplished with a high-efficiency tunnel junction, which converts holes to electrons, extracting two lasing photons for every injected electron. This also requires 10-V voltage compliance from the CMOS chiplets, since the bandgap increases with shorter emission wavelength and the laser diode turn-on voltage is doubled. Only multi-junction VCSELs at 940-nm wavelength are presently commercially available partially due to the challenge of fabricating tunnel junctions in semiconductors with relatively large bandgap energy. The development of the dual-junction short-wavelength VCSEL epitaxy will be pursued with the same foundry, making these high-power VCSELs available in Phase 2.

4.3. Waveguides and Metasurfaces Combined with SLMs to Compensate for Scattering with Wavefront Shaping

The present disclosure's focus on light deep inside a scattering tissue is to launch a dense array of light waves with a high numerical aperture (NA) and tunable optical phases across the array into the tissue. A high NA is crucial because the number of accessible high-transmission “eigenchannels” within a strongly scattering medium is proportional to NA²; the tunable phases will then allow for controlling the constructive interference between these accessible eigenchannels to synthesize a focal spot (with controllable location and size) in the scattering tissue.

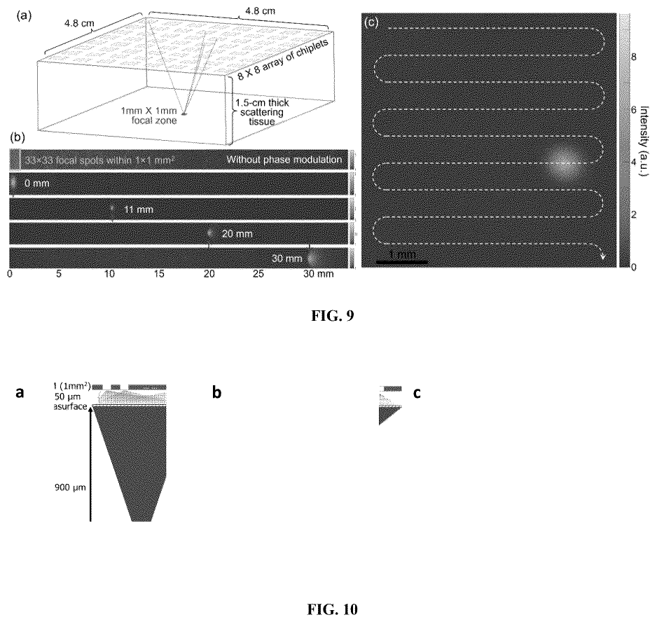

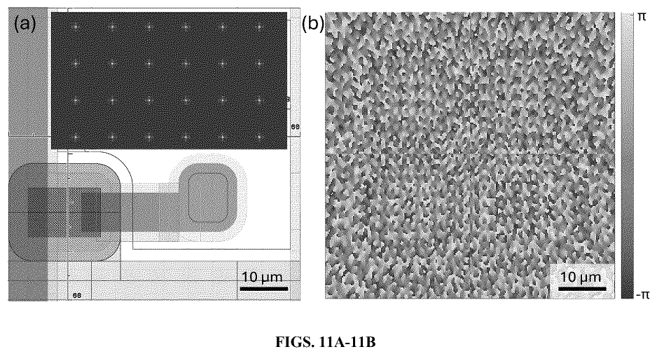

The NIR-HEAD device uses an ultra-thin (<200 μm) optical waveguide-metalens chiplet to couple light from the VCSELs mounted on each CMOS chiplet into a transmissive SLM that covers the entire array (FIG. 14). Within each waveguide-metalens chiplet, a waveguide tree (a network of optical waveguides that branches from a single input to multiple outputs) is used to route and evenly distribute the output from one VCSEL into 16×16 terminal waveguides over an area of 1 mm×1 mm; an array of 16×16 metalenses then converts the emissions from the terminal wave guides into 64×96 high-NA focal spots as inputs for the SLM, with an array of 4×6 focal spot aligned with one pixel of the SLM (FIG. 11a). Compared to the single divergent beam approach pursued in Phase 0 (FIGS. 10a, b), the tree architecture (FIG. 10c) enables the interposer thickness to be reduced by ˜4× (i.e., from 500-700 μm to 100-200 μm). Further, the tree architecture enables a much wider set of fabrication tolerances for achieving uniform intensity and phase among the focused spots. Finally, using the metasurface instead of the VCSEL as the carrier substrate for the interposer scaffold, the present disclosure enables directly oxidization of the scaffold, thereby avoiding the technical challenge encountered in Phase 0 of needing a floating oxidized scaffold. The present disclosure describes the design and fabrication of each of these components (waveguide trees, metalens arrays, SLM). Phase 0 results and plans for the Phase 1 design are summarized below.

### Waveguides, Lenses, Metasurfaces, and SLMs for Light Structuring in the NIR-HEAD Device

### Phase 0 Results

In Phase 0, the present disclosure relied on a vertical lens-waveguide to shape the emission from one VCSEL into a divergent beam and then utilized a metasurface, functioning as a flat optical hologram, to convert the divergent beam into an array of 32×32 high-NA focal spots aligned with the pixels of a liquid-crystal display or LCD (Sony LCX 036) with a 31.25-μm pixel pitch (FIG. 10b). The pair of optical polarizers were removed from the front and back surfaces of the display to convert it to a transmissive SLM. This SLM is only able to produce a phase shift of up to ˜0.67π or ˜120° for the 850-nm light at the normal incident angle.

The metasurfaces, had a linear dimension ranging from 2 to 3.5 mm and were composed of 2D arrays of silicon nano-pillars with a pitch of approximately half of the wavelength (425 nm for 2=850 nm). The nano-pillars had the same height of 700 nm and their diameters were chosen to vary between 100 nm and 290 nm in order to locally change the effective refractive indices. In this way, the metasurface can finely sample and implement the holographic phase profile required for converting the output from the vertical lens-waveguide into the high-NA focal spot array.

The Sony LCD the present disclosure used as an SLM has a 2-mm thick cover glass on either side of the liquid-crystal layer, which introduces two considerable drawbacks: (a) the front cover glass restricts the close proximity between the metasurface and the liquid-crystal layer of the display, limiting the attainable NA of the focal spots (e.g., NA of ˜0.32 for metasurfaces with a diameter of 2 mm and an in-air focal distance of 3 mm); (b) the phase-modulated focal spots become divergent beams in the back cover glass and these beams spatially mix during their propagation through the 2-mm thick glass, reducing the effectiveness of spatial modulation imposed by the LCD. In Phases 1 and 2, the flexible, ultra-thin membranes used to encapsulate the liquid-crystal layer in the transmissive SLMs will solve both problems.

Despite the challenges associated with the limited phase modulation and the thick cover glass of the Sony LCD, the present disclosure experimentally demonstrated focusing of 850-nm near-infrared light from a diode laser through various scattering media; in particular, focal spots with peak intensity 5-7 times higher than the diffusedly transmitted background were observed in experiments with a 15-mm thick skull phantom, which has with a scattering coefficient of ˜0.8 mm-1, similar to that of the mouse skull tissue.

### Design and Fabrication of the Waveguide and Metasurface Chiplets for the Phase 1 Design.

In Phase 1, the present disclosure will design and fabricate the integrated waveguide tree-metasurface array chiplet (FIG. 10c). The emission from the terminal waveguides of one waveguide tree will be an array of 16×16 optically coherent diverging beams with a pitch of 62.5 μm. The present disclosure will scale up production of the interposer chiplets by utilizing Irradiant 3D point-scan lithography systems that have an order of magnitude or more improvement in key performance specifications. The wider field of view will reduce the number of stitches and thus improve the loss and uniformity, while the much faster volumetric print speed is needed to meet program deliverables. The present disclosure will design the metasurface lenses to achieve a moderately high NA of ˜0.68, considering that the metalens diameter is 62.5 μm, the focal distance is 60 μm (i.e., thickness of the tricellulose acetate or TAC covering membranes of the SLMs), and that the TAC has a refractive index of 1.475. Crucially, the focused beams can pass through the actively tunable liquid-crystal windows of the transmissive SLM, acquiring the proper phase modulation without attenuation and avoiding heating the control electronics. Furthermore, with a short propagation distance through the 60-μm thick TAC back cover of the SLM, the focused beams would not spatially mix (as in the Sony LCD); thus, an array of high-NA waves with a high degree of spatial phase modulation can be injected into the scalp to synthesize the focal spot in the brain tissue.

### Design of the SLM for the Phase 1 Design

Two versions of the SLM will be manufactured, one for the rat (8 mm×8 mm; 128×128 pixels) and the other for the human and large animal (46 mm×46 mm; 736×736 pixels). These designs will be twisted nematic (TN) LCDs using organic thin film transistors (OTFT) on flexible 60-μm-thick tricellulose acetate (TAC) substrates. The SLMs will be designed to deliver the full 2π phase shift at λ=650 nm with a pixel pitch of 62.5 μm. For a nematic liquid crystal (LC) with a birefringence of Δn=0.1, (0.15) to achieve the required 2π phase shift will require a cell gap of greater than d=λ/Δn=6.5 μm. The actual gap thickness required will be larger than this because the LCs near the electrode interfaces are not fully rotated. The rotational viscosity of the LCs will limit the SLM frame rate to ˜100 Hz (10 msec per frame), which will be sufficient. Because the SLMs will operate on one specific polarization state, a linear polarizer will be used on one of the surfaces. TAC has extremely low birefringence, making it perfectly suited for use with polarization-sensitive applications. TAC also has extremely low stress birefringence and, as a consequence, will not add any phase retardation when bent or flexed. FIG. 11a shows the 62.5×62.5-μm pixel design, which will be fabricated by the 8-μm lithography capabilities. This pixel design presents a 20-μm-by-40-μm LC window, more than adequate for passing and modulating the focused beams produced by the metalenses (FIG. 11a). Fiducials will be provided on the SLM to allow it to be aligned by the assembly vendor, Promex, with 5-μm resolution to the underlying metasurface-waveguide interposer. The present disclosure will use two commercial off-the-shelf (COTS) driver ICs that are able to drive 640 gate lines (±20 V) and 1000 source lines (+7 V). These chips (each of dimensions 42 mm×20 mm) will be chip-on-flex (COF) packaged onto the SLM substrate. A zero-insertion-force (ZIF) connector will be used to connect the SLM to the underlying rigid board, which will contain the remaining drive electronics as shown in FIG. 14.

In some exemplary embodiments, the current display driver electronics consist of a Black Rev C connected to a FPGA board. The Black Rev C is used to generate 24-bit RGB data (8 bits per pixel), which the FPGA then converts to three-pair mini-LVDS. The FPGA also generates the timing signals for the gate driver IC. The FPGA board features a PMIC that is used to generate the high-voltage supplies required by the display and driver ICs. The present disclosure will instead drive the SLM from the Cortex-A72 on the NIR-HEAD rigid board. The Xilinx FPGA on the rigid board, which is used for the TD-DOT measurement capabilities of the device, will also be used to replace the function of the FPGA board in creating the interface to the driver ICs.

### 4.4. Electronics of the NIR-HEAD Design

CMOS chiplets containing the laser drivers and predrivers and incorporating the time-domain measurement capabilities (described in § 4.5), fabricated in a 55-nm CMOS technology, are mounted in an array on a flexible polyimide PCB. Direct integration of the VCSELs on the surface of the CMOS chiplets helps to make the most compact design possible while also reducing inductive parasitics associated with the interconnection to the drivers, enabling pulsed operation for the time-domain measurement capabilities. A rigid board, connected to the flex board, contains all the additional support electronics. Below summarizes electronics design considerations for NIR-HEAD.

### Electronics Design Considerations for NIR-HEAD

### Phase 0 Results

The present disclosure has already assembled an early version of the AWARE patch with the existing Gen2 chiplet design using the 850-nm custom chiplets developed in Phase 0. In the Gen2 design, the laser drivers are limited to a compliance voltage of approximately 3 V and the anode connection to the VCSELs is made with a wirebond connection.

### Gen3 Chiplet Design

In Phase 1, the present disclosure will re-tape-out the Gen3 chiplet design, which will increase the voltage compliance on the VCSEL drivers to 10 V, allowing support of the high-power VCSEL designs described in § 4.2. At the same time, the present disclosure will fabricate copper pillars for the anode connections to the VCSEL, allowing this interconnection to be made without a wirebond through a metal trace fabricated directly on the waveguide-metasurface interposer as shown in FIG. 14. As part of risk mitigation, the present disclosure is likely to also add support for two additional VCSELs on the Gen3 chiplet as well as perform enhancements to the off-chip digital interface to require fewer interconnections to support denser integration onto the flexible package.

### Rigid-Board Support Electronics

A wireless interface is required to download the TD-DOT data for device calibration and to provide the appropriate phase configurations for the SLM. This wireless interface will rely on the Broadcom BCM2711, Quad core Cortex-A72 (ARM v8) 64-bit SoC and the Infineon AIROC™ CYW43455 WiFi chip, which are the same processor and WiFi chip used in the Raspberry Pi 4. The CYW43455 support 5-GHZ WiFi 5 (802.11ac), which delivers a data bandwidth of 200 Mbps. The present disclosure will leverage the software infrastructure from the Raspberry Pi for this interface. The BCM2711 will interface to a Xilinx FPGA, which will be used to control the NIR-HEAD patch and format the data for transfer between the patch and the BCM2711.

### 4.5. Time-Domain Measurement Capabilities, Reconstruction, and Calibration

This capability builds from previous designs^{6,7,12}, i.e., a flexible patch for NIR imaging of hemodynamics in the brain, which has a very similar form factor to the NIR-HEAD device that leverages the same CMOS chiplets. Each of the NIR-HEAD chiplets is capable of performing time-domain measurement of back-scattered light. In this case, the VCSELs are pulsed one-by-one with a pulse width of 2 nsec at repetition rates as high as 50 MHz. Each array of 20×20 SPADs on a chiplet has 20 dedicated event-driven time-to-digital converters, which together support detection and time-of-flight measurements. SPADs are operated by in-pixel quench-and-reset circuits. The SPAD arrays of all the chiplets simultaneously measure the time-of-flight of detected backscattered photons, resulting in histogram distributions as shown in FIG. 13b, synchronized to the laser pulses as described in Paragraph [0235]. The decay of this histogram when collected for all the VCSELs allows an image of the “baseline” μₐ and μₛ′ as a function of position to be created through inverse imaging algorithms based on non-coherent equations of radiative transfer (ERT). In addition, this TD-DOT measurement capability ensures that all the lasers of the NIR-HEAD device are properly coupled to the scalp optically. In particular, the present disclosure use PHOEBE¹³ to process the data from the TD-DOT measurement hardware to calculate the scalp coupling index (SCI).

For each source location, the present disclosure calculates the SCI to all of the detectors. The photon count as a function of time trace is filtered to the frequency band from 0.5 Hz to 2.5 Hz that includes the heart rate component at around 1 Hz. After normalizing each channel to its standard deviation, one then computes the cross-correlations. Correlations close to one indicate good source (and detector) coupling. This can be used to debug situations in which one or more optical sources are not in good contact with the skin, causing inefficient coupling of light into the scalp. Control of the device and reading of measurement data will occur through the wireless interface described in § 4.6. Below summarizes some of the key progress and design decisions for measurement capabilities on the NIR-HEAD device.

### NIR-HEAD Measurement Capabilities and Dynamic SLM Turning.

### Phase 0 Progress

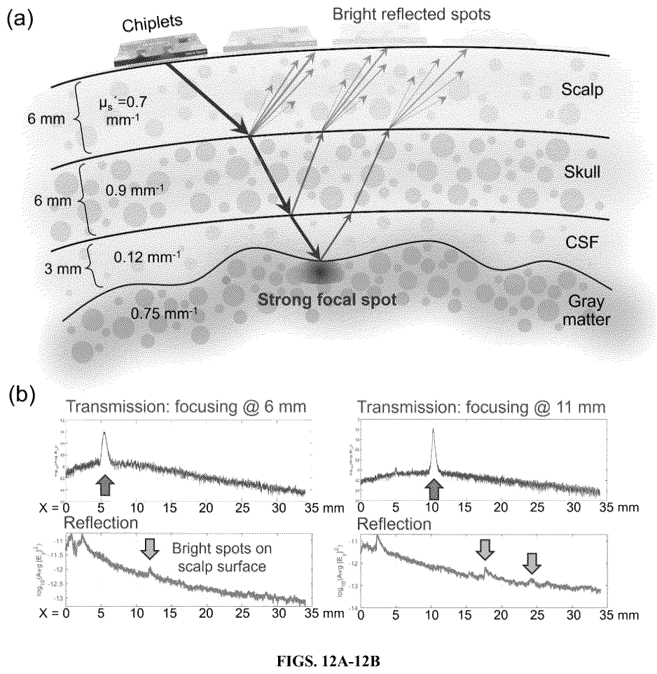

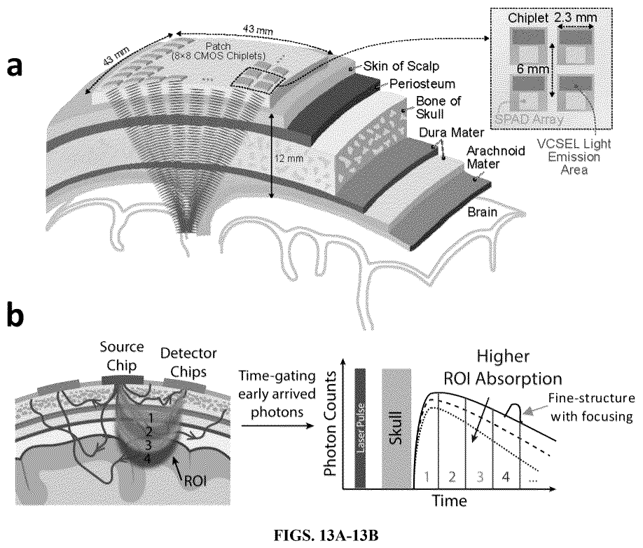

A key challenge of the program is to rely on back-scattered light, on the surface of the scalp, to determine if a focal spot is formed in the brain, and if so, the location of the spot. The present disclosure conducted systematic computational study of light focusing through a thick scattering tissue at various lateral distances from where phase-modulated light waves are injected into the tissue (i.e., position of the VCSEL); in particular, the present disclosure investigated the correlation between forward and backward scattered light inside the tissue. The present disclosure observed that the formation of a focal spot (as a result of proper spatial phase modulation of the injected waves) is always associated with a few bright spots (brightness much higher than the background) on the tissue surface, and that these reflected spots are distributed over a range roughly twice of the lateral distance of the focal spot (FIG. 12). This result suggests an intriguing “specular reflection” in the scattering medium where the contrast between layers of tissues with distinct scattering coefficients and effective refractive indices can reflect light to form multiple bright spots over the input plane (FIG. 12). It was believed that the presence and location of the reflected spots (which can be detected by the SPAD arrays), together with time-resolved back-scattered optical pattern, can be used to predict the presence and location (both lateral position and depth) of the focal spot in the brain.

### Dynamic SLM Adjustment Based on Backscatter Measurements

In Phase 1, the present disclosure will use these bright reflected spots to guide tuning of the SLM, performing these measurements simultaneously with the time-domain measurements of the chiplet. In this case, these reflected spots will be observed at certain locations as shown in FIG. 13b, and the photon arrival time information will be observed in the histograms, allowing for further discrimination of the path depth of the reflection features. This information will allow the optical backscattering measurements to be used to facilitate focusing the light in the forward direction since the magnitude of these reflection features will be related to the quality of the focusing.

### 4.6. Phantoms and In Vitro Testing

The present disclosure employs phantoms for testing the NIR-HEAD device, which follow closely those used previously for NIR brain imaging^{14,15}. The phantoms are characterized by μₐ and μₛ′. In human imaging, inhomogeneities, such as those produced by blood vessels, may affect these results, which will not be reflected in phantom experiments. The present disclosure uses these phantoms to also evaluate tissue heating, which is difficult to do in vivo. Below describes the phantom development.

### Phantom Development.

### Phase 0 Progress

In Phase 0, the present disclosure constructed phantoms made of polydimethylsiloxane (PDMS) with the exception of the gray-matter (GM) layer. The latter is a liquid layer, a mixture of DI water, Intralipid 20%, and India ink. Intralipid is an emulsion of soybean oil that has well-characterized scattering properties. India ink serves as the absorber. DI water is used to control the dilution of the Intralipid. Using a preformed mold and then pouring the various mixtures in one at a time to cure allows one to realize almost any layered configuration. The PDMS tissue layers are made by incorporating specific amounts of titanium dioxide (TiO₂) and India ink into the PDMS-making process. TiO₂ is a particulate that provides scattering in the finished phantom and has almost no absorptive properties. India ink is suspended carbon black, which acts as a very strong absorber with almost no scattering contribution. TiO₂ powder is mixed in the curing agent, and India ink is stirred into the elastomer. These phantoms have been used to characterize our capability to focus NIR light through scattering media in Phase 0.

### Heating-Accurate Phantoms for Phase 1

In Phase 1, the present disclosure intends to use these phantoms to also bound heating. While phantoms lack blood flow, which helps to provide additional heat sinking, they can be used to estimate heat capacity and thermal resistance effects in tissue. To accomplish this, the present disclosure will replace the GM layer with a hydrogel to provide a more tissue-like heat capacity. This will also make it easier to position thermocouples for temperature measurement.

### 4.7. In-Vivo Testing of the NIR-HEAD Design.

In-vivo testing will serve to validate and optimize the TA2-developed NIR light delivery devices and establish safe and effective parameters for the integration with photoactivated pharmaceuticals. These studies are summarized in Table 8.

### Table 8. In-Vivo Testing Plan

### Rat Studies

In Phase 1, studies will use back-scattered light from the wireless 2×2 chiplet device mounted on the rat head to characterize NIR illumination and focusing through the skull and cortex as a function of depth. The present disclosure will support terminal studies to examine the effects of the drug alone and the drug in the presence of focused illumination from NIR-HEAD on neural activation and neural damage. The present disclosure will be focused on demonstrating that NIR light with an irradiance of at least 4 mW/mm² does not activate the prefrontal cortex (PFC). Animals (n=24; n=6 for each of the wavelengths of control, 680 nm, 780 nm, and 850 nm) will be treated with 90 minutes of NIR light before being sacrificed for terminal comparisons of the activation of the early immediate gene c-Fos in PFC tissues prepared via immunohistochemistry. A separate cohort (n=24) will be treated similarly but analyzed for whole brain c-Fos using iDisco to allow for examination of neural activation patterns in structures outside of the PFC that are implicated in the potential off-target effects, such as mesolimbic circuitry (e.g., ventral tegmental area and nucleus accumbens) and sleep circuitry (e.g., hypothalamus, locus coeruleus). The 90-min timepoints chosen for these studies reflect the temporal dynamics of peak in-vivo c-Fos activation. Parallel studies will employ fiber photometry recording of GCaMP fluorescence in the rat PFC to measure real-time calcium dynamics. These recordings (n=30) will capture the kinetics, amplitude, and temporal pattern of neural activation, providing a continuous measure of cortical activity that complements terminal c-Fos histology.

To provide additional safety evidence necessary to advance NIR-HEAD device toward human application, the present disclosure will implant rats (n=12) with telemetric transponders and continuously monitor physiological parameters including heart rate, core body temperature, and activity throughout six hours of NIR stimulation. Blood samples taken throughout testing will be taken for ELISA analysis of plasma corticosterone, norepinephrine, epinephrine and immune markers (e.g., cytokines). To ensure that illumination parameters necessary for photopharmaceutical activation have no adverse effects on brain tissue, rat PFC will be exposed to NIR for six hours before animals (n=6 control and n=6 NIR) are sacrificed for immunohistochemical analysis of tissue damage (e.g., Nissl stain, H&E, NeuN), neuroinflammation (e.g., GFAP, Ibal), and DNA fragmentation (e.g., Terminal deoxynucleotidyl transferase dUTP nick end labeling). Together, these measures allow for comprehensive evaluation of whether NIR illumination elicits unwanted neuroendocrine or immune effects, ensuring that illumination parameters are physiologically safe and non-disruptive.

### Porcine Studies

The large animal pre-clinical studies will be performed using a swine model. Here, the present disclosure aims to implement, validate, and optimize the 8×8 human device. The preliminary goal is to assess the safety of the devices and the potential impact NIR may have on the PFC of swine. These studies will subsequently integrated to the drug developed. In Phase 1, swine (n=8, 50% males and 50% females) will be equipped with the wireless 8×8 devices and n=4 animals will undergo NIR and n=4 will serve as controls. The present disclosure will focus light in the cortex and use the measurement capabilities of the device to infer delivered power and depth based on back-scattered light. The present disclosure will ensure that illumination at the focused target irradiance of 4 mW/mm² provides no noticeable phototoxicity or neuromodulatory effect in the PFC. Here, target depths will be 1.5 cm (superficial cortex) with illumination volumes up to 1 cm³ through raster scanning. The present disclosure will also test the off-target effects of the illumination on the brain distal to the PFC, as well as on pig physiology.

Specifically, the present disclosure will examine the effects of NIR illumination on the animals' heart rate, blood pressure, temperature, respiratory rate, as well as gait and activity levels. Blood draws will be taken to examine plasma corticosterone, cell blood counts, serum chemistries, and immune cell populations via flow cytometry. Both myeloid (monocytes/granulocytes) and lymphocyte populations (effector/activated T cells, regulatory cells and B cells) will be analyzed. All samples will be taken before and after 8 hours of NIR illumination. Inflammatory cytokines will be performed with a swine cytokine array. PFC phototoxicity in recipients undergoing NIR treatment will be assessed post-mortem via immunohistochemical analyses of brain damage (e.g., Nissl stain, H&E, NeuN), inflammation (e.g., GFAP, Ibal), and cell-activation (e.g. c-Fos) similar to what will be done in the rat model. The six-hour timepoint aims to translate to what human exposures may be over one session.

To provide additional safety evidence necessary to advance the TA2 NIR device toward human application, the present disclosure will implant rats (n=12) with telemetric transponders and continuously monitor physiological parameters including heart rate, core body temperature, and activity throughout six hours of NIR stimulation. Blood samples taken throughout testing will be taken for ELISA analysis of plasma corticosterone, norepinephrine, epinephrine and immune markers (e.g., cytokines). To ensure that illumination parameters necessary for photopharmaceutical activation have no adverse effects on brain tissue, rat PFC will be exposed to NIR for six hours before animals (n=6 control and n=6 NIR) are sacrificed for immunohistochemical analysis of tissue damage (e.g., Nissl stain, H&E, NeuN), neuroinflammation (e.g., GFAP, Ibal), and DNA fragmentation (e.g., Terminal deoxynucleotidyl transferase dUTP nick end labeling). Together, these measures allow for comprehensive evaluation of whether NIR illumination elicits unwanted neuroendocrine or immune effects, ensuring that illumination parameters are physiologically safe and non-disruptive.

### 4.8. Technology Challenges

Technical challenges and risks in the design of the NIR-HEAD device are noted in the attached Risk Matrix along with proposed mitigation measures. Phase 1.

In this phase, the present disclosure will bring the NIR-HEAD device to the point of small-scale manufacturability by engaging vendors for the development and manufacture of a mechanically flexible LCD-based spatial light modulator, the manufacture of VCSELs at five different wavelengths between 650 nm and 850 nm, the manufacture of the metasurfaces, the manufacture of the waveguides and lenses for the VCSELs, and the assembly of the NIR-HEAD patches. Manufacturing will take place in a manner that conforms with ISO 13485 for GMP manufacturing, which will be required for FDA approval. In some exemplary embodiments, the human and large animal version will consist of an 8×8 array of chiplets on a 6-mm pitch, building on the existing design used in Phase 0, which is a 4×4 array of chiplets on an 8-mm pitch. In addition, in some exemplary embodiments, the present disclosure will develop a version of the device for the rat, which will consist of only four chiplets on a 6-mm pitch. Table 1 summarizes the devices developed, the characteristics of these devices across different versions, and the number and availability of these devices. The VCSELs will be developed in the program to cover all possible wavelengths. Other components, notably the waveguide chiplets and SLMs, will be produced in quantities to support fabrication on the device numbers shown in Table 1 at a single target wavelength to control manufacturing costs.

1.1. NIR-HEAD patch development. The present disclosure will bring to small-scale manufacturing the design of two patches, the 8×8 for human and large-animal use and the 2×2 design for the rat model. This includes the design of a modified Gen3 chiplet design, new VCSEL development, design of lenses and metasurfaces to focus this light into the SLM pixels, and a custom SLM design.

1.1.1. Gen3 chiplet development. The present disclosure will perform a single design respin of the current Gen2 chiplet design used in Phase 0 to enable support of a larger compliance voltage for the laser drivers. This reflects the fact that the original CMOS chiplets were not designed for this application and have a limited 2.5-V bias for the VCSELs. At the same time, the present disclosure will reposition the anode contact for VCSEL connections to enable Cu pillar processing to allow these top connections to be made without the need for wirebonds, which will ease the assembly of the VCSEL and metasurface-waveguide chiplets. The present disclosure is scheduling the tape-out of this respin for the first month of Phase 1 but hope to tape this out in Phase 0. The Gen3 chiplets will be bump compatible with the Gen2 design, allowing all of the interfaces and packaging to remain the same between Gen2 and Gen3.

| NIR-HEAD | CMOS | VCSEL |
|---|---|---|
| version | chiplet version | wavelengths/powers |
| 2 × 2 (Ver 1) | Gen2 | 680 nm (2 mW), 780 nm (5 mW), |
| | | 850 nm (5 mW) |
| 8 × 8 (Ver 1) | Gen2 | 680 nm (2 mW), 780 nm (5 mW), |
| | | 850 nm (5 mW) |
| 8 × 8 (Ver 2) | Gen3 | 650 nm (2 mW), 700 nm (2 mW), |
| | | 750 nm (5 mW), |
| | | 800 nm (5 mW), 850 nm (5 mW) |
| [symbol] | [symbol] | [symbol] |

[symbol]

indicates data missing or illegible when filed

Approach: The present disclosure will perform a re-tape-out of the Gen2 chiplet design to increase the compliance voltage to 8 V for the VCSEL drivers. This is possible because the technology employed in this design (GF 55 nm BCD) process supports high-voltage devices for this function. Wafers from GF will be bumped by the vendor Pactech for the peripheral connections of the chiplet to the flexible package with a Cu pillar fabricated for the two anode contacts to the VCSELs. The wafers will then be thinned and diced by the vendor QPTechnologies. All of the designs tested in Phase 1 will use the existing Gen2 chiplets; the present disclosure will move to the Gen3 chiplets in Phase 2 to support the high-power laser designs.

1.1.2. VCSEL development. The present disclosure will ultimately develop custom single Gaussian mode VCSELs that are able to deliver at least 5-mW continuous wave at target wavelengths between 650 nm and 850 nm. The use of single-mode VCSELs helps to ensure that the pattern of illumination out of the VCSEL aperture does not change with bias or temperature, which will be important for the design of the lens, waveguides, and metasurfaces that structure this light for delivery into the SLM. VCSELs will be manufactured to the custom specifications for the NIR-HEAD device.
Approach: For initial development, the present disclosure will employ existing epitaxial wafers available at DQD at wavelengths of 680 nm, 780 nm, and 850 nm. VCSELs can be pad and form factor compatible with both the Gen2 and Gen3 CMOS chiplets of NIR-HEAD, allowing them to be used interchangeably. In some exemplary embodiments, the VCSEL structures are monolithic distributed Bragg reflectors and multi-junction quantum-well core designs. To achieve the 5-mW CW power targets for the shorter (<700 nm) wavelengths, a multi-junction design can be employed. Each VCSEL can be fabricated as a two-VCSEL chiplet designed to be bonded directly to the surface of the CMOS chiplet.
Development by DQD of 780-nm and 850-nm, single-mode, 5-mW VCSELs, along with 680-nm, 2-mW, single-mode VCSELs in quantities of at least 720 for each type, which is sufficient for the delivery of 20 2×2 patches, and 8 8×8 patches for large-animals.
Completion of the epi and progress toward fabrication of the 650-nm, 700-nm, 750-nm, 800-nm, and 850-nm VCSELs by DQD. 2-mW CW power targets will be achieved at 650-nm and 700-nm with 5-mW CW power targets achieved for the other wavelengths.
Development of the epi for 650-nm and 700-nm multi-junction VCSELs to achieve 5 mW single-mode power targets

1.1.3. Metasurface and lens manufacturing. The present disclosure will execute small-scale manufacturing of VCSEL-to-metasurface optical interposer chiplets. Each chiplet will be an oxidized porous film directly bonded to the metasurface. Each chiplet includes a VCSEL to waveguide coupling lens and a compact waveguide splitter tree that will generate a 16-by-16 grid of expanding spots. These spots, which span the entire 1-mm² area, will be directly manipulated by the metasurface and spatial light modulator (SLM) pixels. During assembly, the interposer chiplet will be positioned over the VCSEL aperture to collect the VCSEL output. The output waveguide for each leaf of the splitter tree will have a graded index and thereby create a 16-by-16 grid of expanding beams using the techniques developed in Phase 0. The metasurface will focus these expanding beams to create large emission angles (NA˜0.6-0.7), to fully access open eigenchannels in the scattering medium. A custom-developed transmissive SLM developed in 1.1.3 will be used to modulate the phase of each spot independently, to ensure constructive interference between the open eigenchannels, such that maximal optical power can be delivered to a target spot in the scattering medium. The metasurfaces will be manufactured at NILT using nanoimprint lithography for high-throughput fabrication. These metasurfaces will be designed for all of the possible target wavelengths. As much as possible, the present disclosure will attempt to begin some of this development in Phase 0.
Approach: The development of the interposer and metasurface chiplets, and their experimental testing using the phantoms in 1.2 can be divided into the following steps as developed in Phase 0. (a) The graded-index lens and waveguide tree is designed and fabricated to create a 16-by-16 grid of expanding beam spots spanning 1 mm² at a distance of 200 μm above the VCSEL. The transverse mode (amplitude and phase distributions) of the VCSEL emission at a specific driving current is characterized to optimize lens design. (b) A metasurface is designed and fabricated to convert each of the 16-by-16 beam spots into 4-by-6 diffraction-limited focal spots at a distance of 60 μm on the transmission side of the metasurface, which is sufficient to reach the liquid crystal center of the SLM pixels. The metasurface is composed of an array of silicon nitride nanopillars with subwavelength dimension and subwavelength pitch. The diameter of each nanopillar will be controlled to introduce a local phase delay so that collectively the 2D array of nanopillars will provide a smooth phase modulation for the conversion operation. (c) The array of 64-by-96 optical spots is phase modulated by an array of 16-by-16 pixels of the transmissive SLM with a pixel pitch of 62.5 μm: each subarray of 4-by-6 spots acquires an independent phase tuning upon transmission through the active region of a pixel of the SLM. The phase modulation of the SLM is optimized to maximize the optical intensity at a target spot on the transmission side of the phantom. (d) Two VCSELs from the same chiplet and, subsequently, VCSELs from multiple chiplets, with the assistance of the graded-index lenses, splitter trees, metasurfaces, and SLM, can combine their output to maximize the optical intensity at a single target spot on the transmission side of the phantom. The present disclosure will manufacture the metasurfaces at-scale at NILT and the interposer chiplets at-scale. The metasurfaces will be incorporated onto an optical spacer layer fabricated from a flexible polymer, such as polyethylene (PE) or polycarbonate (PC). These same substrates, upon which the metasurfaces are fabricated, will form the support for the lens and waveguide structures. The lens and waveguide structures fabricated in Phase 1 will be micromachined to accommodate the wirebond anode connections to the VCSEL Gen2 chiplets.

1.1.4. Manufacture of the transmission SLM. The present disclosure will develop custom SLMs for both the 2×2 and 8×8 NIR-HEAD devices.
Approach: These designs will be mechanically flexible, TN LCD displays with a pixel pitch of 62.5 μm that will deliver at least 2π phase shift at 650 nm. Electronics for the driving of this display using two custom chip-on-flex (COF) driver ICs will be incorporated onto the flexible display board using anisotropic conductive film (ACF) bonding; additional driver electronics will be contained on the rigid circuit boards developed in 1.1.5, which will connect to these SLMs. The array size for the 2×2 chiplet design will be approximately 8 mm×8 mm (128×128 pixels); the flexible board itself will expand out to a width of 46 mm to accommodate the source and gate COF driver ICs and the connector to the 2×2 version of the rigid board. The flexible board for the 2×2 array developed in 1.1.5 will have a similar form factor. The array design for the 8×8 chiplet design will be approximately 46 mm×46 mm (736×736 pixels), but the board extends an additional 10 mm to accommodate the COF driver ICs on the same side as the connector to the 8×8 version of the rigid board.

1.1.5. Printed-circuit board manufacturing. The present disclosure will develop both flexible and rigid printed-circuit boards (PCBs). The flexible PCBs will accommodate the integration of the chiplets and allow the array to conform to the contours of the head. The rigid PCBs will accommodate all of the commercial off-the-shelf chip sets, including the wireless interface, the power management, the FPGA for signal processing, and the processor. The present disclosure will manufacture two distinct flexible boards, one to support the 2×2 chiplet design and one to support the 8×8 chiplet design. These will be manufactured by ETEC and will support both the integration of the chiplet array and the assembly of all of the components needed to support the data acquisition and wireless interface. The rigid boards will be designed at a commercial PCB vendor. Assembly will be done as part of the overall patch integration in 1.1.5.
Approach: The 2×2 and 8×8 flexible boards will use ENEPIG plating to support direct flip-chip attached of the Gen2 or Gen3 chiplets. The rigid boards will include the COT components to support the signal processing, power management, and wireless interface. A zero-insertion-force (ZIF) connector will be used to connect the flexible board to the rigid board. An additional ZIF connector will allow the SLM developed on 1.1.4 to also be connected to the rigid board.

1.1.6. Patch integration. The components of the patch will be assembled according to ISO 13485 standards to meet FDA GMP requirements for the 8×8 chiplet. The 2×2 chiplet devices can be used to develop the process and gauge the achievable alignment tolerances. This work includes the assembly of the rigid printed circuit board design 1.1.5 and all the integrated components on this board as well as the assembly of the flex board and integration with the SLM. These devices will be assembled with the VCSELs developed in 1.1.1 at wavelengths of 680 nm, 780 nm, and 850 nm
Approach: The rigid PCBs will first be assembled. Assembly of the flexible PCBs will involve the following steps:
   Bump-bonding attachment of the CMOS chiplets to the flexible board.
   Attachment of the VCSEL chiplet to the CMOS chiplet. Connection for the anode is made with conductive epoxy. In Gen2, an additional wirebond connection is made between the anode and the CMOS chiplet that is not necessary for the Gen3 design.
   Alignment and attachment of the waveguide-metasurface chiplet to the VCSEL. This is the most critical alignment step requiring sub-micron-scale alignment. Fiducials will be provided for this alignment, but the present disclosure will work with the assembly vendor to perform active alignment with the VCSELs operating. Attachment will be with a UV-curable epoxy.
   Alignment and attachment of the SLM. This requires 5-μm alignment tolerances to ensure that the focal spots created by the metasurfaces are centered on the liquid-crystal “open window” of the pixel. Attachment of the SLM to the patch will also be with a UV-curable epoxy; the SLM will be in direct contact with the “top” of the wavguide-metasurface chiplet.

1.2. Phantom testing of NIR-HEAD device and studies of expected anatomical variation.

This task includes the development of the phantoms, the development of the TD-DOT construction of these phantoms, and verifying the degree of focusing and power densities achieved. Metasurfaces will be augmented with the same commercial SLM used in 1.2 to allow for reconfigurability; this testing will form the basis for a custom SLM design in Phase 1.

1.2.1. Study of expected human anatomical variation and development of phantoms that cover these variations. These phantoms will also be constructed to allow accurate measurement of heating; heating analysis in the absence of vasculature will be a “worst case” assessment of heating conditions.
Approach: These phantoms will build on those constructed in Phase 0. In some exemplary embodiments, in Phase 0, the present disclosure constructed a set of “flat” phantoms that consisted of four compartments: three lower, solid compartments and an upper, liquid compartment. The lower compartments of the phantom are made with cured PDMS containing a specific mixture of titanium dioxide (TiO₂) particulates and India ink that determines the optical scattering and absorption properties of the PDMS. The lower compartments mimic the superficial tissues of the human head: a scalp of 6-mm thickness, a skull of 6-mm thickness, and cerebrospinal fluid (CSF) of 2-mm thickness. The upper, liquid compartment of the phantom was filled with a solution of Intralipid 20% and India ink suspended in distilled water, which has scattering and absorption coefficients roughly equivalent to grey matter (GM) in the brain. The optical properties and thicknesses of the scalp, skull, CSF, and GM layers in the phantom were chosen to be consistent with tissue beneath the Oz landmark in the 10-10 system. In Phase 1, the intralipid solution, may be replaced with an agar/intralipid/India ink molded media that will be easier to manage. The present disclosure will use 3D printed molds to form the solid PDMS portions of Phase-0 phantoms to allow them to have more realistic curvatures. The present disclosure will cover the spectrum of possible human variability that may be observed in prefrontal cortex in these phantoms, developing at least 8 different types. The =present disclosure will also incorporate different levels of absorption in the skin layers to cover the range of skin color variations one would expect to see as well. At the same time, the present disclosure will have the option to position thermocouples within the agar to measure heating as a result of the action of the NIR-HEAD.

1.2.2. Measurement of time-domain backscattered light using the measurement capabilities of the NIR-HEAD device in the phantoms of 1.2.1. The present disclosure r will use the average parameters of the histogram response to perform TD-DOT reconstructions of scattering and absorption in the phantoms. The present disclosure will also extract the fine features in these histograms, which were discovered in simulations in Phase 0, which should allow us to verify focusing and be used to tune the SLMs. Depending on additional progress in Phase 0, these efforts will continue here. These models differ from previous efforts because they consider phase coherence (electric field modelling) as well as consider the effects of the metasurfaces, which are shaping the excitation light.
Approach: Measurement data will be brought off the patches. The results of this analysis will be used to tune the phase shift of the SLMs to focus the light and will also allow us to correct for person-to-person variability. The present disclosure will determine how the properties of the back-scattered light, which is measureable, determine the transmitted light and how the backscattered light measurements can be used to reconfigure the system.

1.3. In-vivo testing of the NIR-HEAD patch design. This task will include both rodent and porcine testing of the NIR-HEAD design.

1.3.1. Rodent testing of the 2×2 NIR-HEAD patch design. The present disclosure will test ability to mount the device on the freely moving rat and perform TD-DOT imaging through the skin and skull. The present disclosure will focus light at various target positions in the cortex and use the measurement capabilities of the device to infer delivered power based on backscattered light. Heating effects will be correlated with the phantom measurements of 1.2.1. This approach is superior to any invasive techniques, which are likely to produce effects that interfere with the phototoxicity and neural activity analysis performed here. The present disclosure will ensure that this light delivery has no noticeable phototoxicity or neuromodulatory effect in the PFC with the use of CW illumination of the magnitude required to photoexcite the photopharmaceutical with the parameters as outlined in Volume 1. Light levels will remain below the ANSI standard for CW light delivery to the skin. In this task, the present disclosure will also provide support in the use of the 2×2 chiplet design in their drug activation experiments. The wavelengths to be employed here (from the available 680 nm, 780 nm, and 850 nm).
Approach: Rodent 2×2 chiplet devices will be fixed to the heads of rats in a position that targets the PFC. Backscattered light will be used to measure power density and irradiance at the target region. Light sources for each of the three available wavelengths (680 nm, 780 nm, and 850 nm) will be tested in a counter-balanced, within-subjects design in n=12 rats. The present disclosure will next examine the effects of illumination (n=6/wavelength) on PFC neural activity by immunohistochemical analyses of the early-immediate gene for cell-activation (e.g., cFos). Animals will be sacrificed after 90 minutes of NIR activation as this timepoint is ideal for capturing changes in cFos activity. A separate cohort (n=24) will be treated similarly but analyzed for whole brain c-Fos using iDisco. To measure effects on physiology, rats (n=12) will be surgically implanted with a telemetry device that allows for continued measurement of core body temperature and heart rate or blood pressure. Baseline measurements will be established and then animals will be administered either six hours of NIR illumination or no illumination. Blood will be sampled throughout for plasma measurements of corticosterone, epinephrine, norepinephrine and immune markers. Rats will be tested within-subjects with each animal receiving both treatments, separated by 72 hours. At the conclusion of testing, the present disclosure will examine the effects of extended (six-hour) illumination on PFC phototoxicity (n=6/condition) by histopathological/immunohistochemical analyses of brain damage (e.g., Nissl stain, H&E, NeuN), inflammation (e.g., GFAP, Ibal), and DNA fragmentation via TUNEL assay. Illumination will be delivered with CW illumination up to the maximum output power for all VCSELs. It is important to note that because these tissues are shallower than in the large animal and human studies, comparable power densities can be achieved with only the 8 VCSELs of the 2×2 chiplet design. This task uses Verl of the 2×2 patch design (see Table 1).

1.3.2. Non-GLP porcine testing of the 8×8 NIR-HEAD patch design. In this task, the present disclosure will test ability to mount the 8×8 NIR-HEAD device (Verl) on the freely moving pig and perform imaging through the skin and skull. The present disclosure will focus light in the cortex and use the measurement capabilities of the device to infer delivered power based on backscattered light. The present disclosure will ensure that CW illumination has no noticeable phototoxicity or neuromodulatory effect in the PFC. Light levels remain below the ANSI standard for CW light delivery to the skin. In this task, target depths will remain 1.5 cm (superficial cortex). The present disclosure will also test the off-target effects of the illumination on pig physiology.
Approach: Porcine 8×8 chiplet devices, specific to a NIR wavelength informed by rodent studies, will be fixed to the heads of pigs (n=8) in a position that targets the PFC. Backscattered light will be used to measure power density and irradiance at the target region during illumination. The present disclosure will also investigate the effects of extended (6-hr) NIR illumination on pig physiology and histopathology. Blood draws, physical exams, and CT scans (blood perfusion) will be performed before and after treatment (6 hrs). Continuous telemetry recordings will sample heart rate, blood pressure, temperature, respiratory rate throughout treatment. Pigs will be treated within-subjects in a counterbalanced design with treatments separated by 72 hours. Blood will be analyzed for plasma corticosterone, cytokines, blood cell counts, and blood chemistries. In a terminal analysis of PFC phototoxicity and neural activity, pigs (n=6/condition) will be treated with NIR or no treatment and sacrificed 6 hrs later for by histopathological analysis of brain damage (e.g., Nissl stain, H&E), assessed by a blinded board-certified veterinarian. Tissues will also be processed for immunohistochemical analyses of cell activation (e.g., cFos), and inflammation (e.g. GFAP, Ibal). This task uses Verl of the 8×8 patch device (see Table 1).

1.4. Preparation for IACUC and ACURO activities for Phase 2 and pre-sub meeting with FDA. In this task, the present disclosure will establish IACUC protocols to perform the porcine studies of Phase 2 and also receive ACURO approval for these studies. The present disclosure will test the light-delivery devices in pigs while coordinating on a second rat protocol and pig protocol. The NIR-HEAD device is a non-significant risk (NSR) device; the present disclosure expect to be able to obtain IRB approval for this device without an FDA NSR determination. This assessment is based on the fact that an IRB for a TD-DOT device operating at similar laser power levels already existed. However, the NIR-HEAD device will be viewed as a combination product by FDA when used in combination with the drug target.

### 1.4.1. IACUC and ACURO Approval for Rat Studies.

Approach: The present disclosure will file for IACUC and ACURO approach to test the four-chiplet, Generation-2 device in the rat model.
   1.4.2. Participate in Pre-IND Discussions with FDA for the Use of NIR-HEAD as a Combination Product.
Approach: The present disclosure will participate in an INTERACT meeting with FDA to understand the testing requirements for a combination product using the NIH-HEAD device.

### Phase 2.

In this phase, the present disclosure will focus on the performance of both non-GLP and GLP porcine studies and deliver 8×8 NIR-HEAD devices that operate with at least 5-mW, single-mode CW power at wavelengths of 650 nm, 700 nm, 750 nm, 800 nm, or 850 nm.

### 2.1. Continued NIR-HEAD Device Development and Delivery.

2.1.1. Continued VCSEL development. In this task, the present disclosure will fabricate VCSELs with the full complement of wavelengths (650 nm, 700 nm, 750 nm, 800 nm, 850 nm). The present disclosure will then bring in the higher-power multi-junction designs for the 650-nm and 700-nm wavelengths that should better support the requirements for stimulation at the larger 30-mm depths.
Approach: DQD will complete the fabrication of at least 500 VCSELs in each of the five wavelengths using the epi developed. After initial prototyping of the epi for the multi-junction designs, this epi will be transferred to DQD for VCSEL fabrication, resulting in 500 VCSELs at each of these two wavelengths.

2.1.2. Metasurface and interposer development. In this task, the present disclosure will manufacture the metasurfaces required to support the additional wavelengths and manufacture the interposer chiplets for the new VCSELs developed in 2.1.1. These will be designed for the Gen3 chiplets and include the additional metallization to allow electrical interconnection from the anode of the VCSEL to the Cu pillar on the Gen3 chiplet design.
Approach: Metasurfaces for wavelengths 650 nm, 700 nm, 750 nm, 800 nm, and 850 nm will be manufactured by AIM Photonics. Interposer/metasurface chiplet will be bonded onto the new VCSELs developed in 2.1.1 in 2.1.3.

2.1.3. Patch integration. The components of the 8×8 patch will be assembled according to ISO 13485 standards to meet FDA GMP requirements using the new VCSEL designs. As part of this task, the present disclosure will also continue to develop the firmwave and software of the NIR-HEAD system.
Approach: the full 8×8 patch will be assembled
These devices are capable of delivering a spatial coverage of at least 1 cm³ with a resolution of 1 mm³ volume at a tunable depth of up to 3 cm below the surface of the skull, as demonstrated using a tissue phantom of human scalp, skull, and brain. Metrics include total illumination volume sufficient for illumination of target PFC subregion(s), as determined in Phase 1; total weight of emitter and associated wiring and electronics<100 g; rechargeable and/or replaceable power supply (e.g. battery)<100 g; total device weight<200 g; power supply (e.g. battery) sufficient for a minimum of 60 min. of continuous operation, with a targeted 4 h of continuous operation, and a reach goal of 40 h continuous operation from a single charge. This device constitutes a “design freeze” for both the GLP and non-GLP testing of 2.2.

2.2. In-vivo studies. In this task, the present disclosure will support in-vivo studies required to submit an IND to FDA at the end of the phase for the NIR-HEAD device as part of a combination product.

2.2.1. Non-GLP porcine studies, the present disclosure will perform non-GLP studies of arousal and sleep in the pig under NIR PFC illumination.

Approach: The present disclosure will support in the investigation of changes in behavior (arousal and sleep). Pigs (n=20) will be outfitted with telemetry. Porcine 8×8 chiplet devices will be fixed to the heads of pigs in a position that targets the PFC. The present disclosure will use video tracking to examine the effects of 6-hrs NIR and/or Photo-Dex on sleep latency and sleep patterns in sleep deprived pigs. Each animal will undergo 24 hours of sleep deprivation before treatment. Conditions will include (1) Vehicle+NIR, (2) Vehicle without NIR. All tests will be conducted with-subjects with treatments separated by 72 hours to allow for sleep restoration. Physical exams, CBC/chemistries and flow cytometry will be performed before and after treatment periods. Video analysis will be used to determine treatment-induced changes in sleep latency and activity. 2.3. FDA interactions. In this task, the present disclosure continues interactions and discussions with FDA recording the NIH-HEAD device as a combination product. The present disclosure submits an IND at the end of the phase in collaboration with a government transition partner.

2.3.1. Participate in Pre-IND Discussions with FDA for the Use of NIR-HEAD as a Combination Product.
Approach: The present disclosure will participate in a pre-IND meeting with FDA to discuss the animal testing protocols to ensure that they will be sufficient to meet regulatory requirements for a combination product using the NIH-HEAD device.
   2.3.2. File an IND Discussions with FDA for the Use of NIR-HEAD as a Combination Product.
Approach: The present disclosure will file an IND application with FDA for a combination product using the NIR-HEAD device.

### Discussion

The goal of this Near-Infrared Head-Mounted Excitation and Activation Device (NIR-HEAD) effort is to develop a wearable device to deliver near-infrared (NIR) light into the brain in a wearable microsystem to activate photopharmaceuticals, i.e., photoactivatable dextroamphetamines. In some exemplary embodiments, efficient delivery of light from an array of vertical cavity surface emitting lasers (VCSELs) into the head is achieved by maximizing the surface area and range of incident angles for optical coupling, which is realized by an ultrathin, flexible optical interposer consisting of an array of wide-branching waveguide trees aligned with an array of high numerical-aperture (NA) metasurface lenses. Focusing NIR light in the brain is achieved through optical wavefront shaping with a transmissive spatial light modulator (SLM). In addition to the final form factor for human use (and large animal studies), a smaller microsystem for optical stimulation at shallower depths will also be produced for rodent studies. The intended use for the device in prefrontal cortex (PFC) eliminates the need for handling hair, which is problematic for these applications due to the potential for absorption and poor coupling of optical emitters to the scalp. The NIR-HEAD device will be able to measure its own optical coupling and provide warning if the device is not in proper contact with the scalp. In addition, the device has the ability to measure time-domain backscattering of the wavefront-shaped light, which enables extraction of not only average parameters (such as absorption coefficients, μₐ, and reduced scattering coefficients, μₛ′, as a function of position) from the diffuse light characteristics but fine features in the temporal and spatial distribution of the back-scattered light, allowing SLM phases to be dynamically adjusted for focusing NIR light into prescribed brain regions across individual variations and in response to temporal changes. The present disclosure has engaged manufacturing partners for the NIR-HEAD device that will allow for small-scale manufacturing according to ISO 13485 standards for good manufacturing practices (GMP) as required by the FDA for human use.

It has long been known that scattering in turbid media can be compensated with wavefront shaping^{2,3}. Approaches to achieve this have relied exclusively on complex, bulky optical systems using reflective SLMs, and measurement approaches to tune these SLMs have relied on access to the transmitted light. With an emphasis on imaging depth, none of these efforts have been directed toward delivering focused light at depth in tissue. In NIR-HEAD, the present disclosure applies the latest in electronic and photonic design, manufacturing, and packaging to develop a true microsystem for focused light delivery, informed by in-situ and real-time measurement via the same device of the temporal and spatial features of the back-scattered light. In addition to the photoactivatable dextroamphetamines considered here, this device could have significant commercial applications for other photopharmaceuticals (e.g., drugs for epilepsy) in which directed optical delivery would be key to reducing off-target effects. NIR-HEAD will also help to advance functional brain imaging applications that are already under development with an eye toward commercialization with a start-up venture.

The constraints on NIR-HEAD are dictated by the requirements that the optical power at the skin cannot exceed the ANSI Z136.1-20224 maximum permissible exposure (MPE) limits as determined by heating. The present disclosure extends these same heating constraints everywhere; in the brain, this allows infrared neural stimulation, which is driven by heating effects, to be avoided⁵. The exposure limit under continuous-wave (CW) conditions is given by 2000×10^{0.002(λ-700)} W/m² for λ between 700 and 1050 nm, which evaluates to 4000 W/m² or 4 mW/mm² at λ=850 nm. If pulsed excitation is used, tolerable instantaneous optical intensity levels can be considerable higher as long as the pulse duty cycle is such that the average power is not exceeded over the time course of the pulse sequence. While CW light delivery will be employed in NIR-HEAD unless raster scanning is employed as described below, pulsed excitation will be used for time-domain measurement and calibration.

Scattering (primarily Rayleigh scattering with 1/λ⁴ wavelength dependence) and absorption are the principal impediments to focusing light to depths of 15 mm or greater. The mean-free path (λ_{mfp}) is about 1.4 mm in the scalp and skull, leading to almost completely diffusive transport by 4-mm depth. The spatial resolution requirements of a 1-mm³ illumination volume at 30-mm depth are the most challenging. The only way to overcome such scattering is with wavefront shaping, which the present disclosure will realize with the use of a transmissive spatial light modulator (SLM). The SLM will shape the wavefronts² to produce as close to a focused beam as possible at the target location at a depth of between 15 mm and 30 mm (FIG. 13a). Individual variations as well as temporal changes in scattering and absorption will affect the quality of this wavefront shaping and must be dynamically adjusted through measurement of the back-scattered light.

TABLE 2
Properties of target photopharmaceutical and focusing
efficiency and achievable irradiances from simulation.
Photopharmaceutical specifications

| Molar extinction coefficient, ε (M⁻¹cm⁻¹) | 30,000 | |
|---|---|---|
| Absorption cross section, σ (cm²) | 1.15 × 10⁻¹⁶ | |
| Quantum yield (Φ) | 0.4 | |
| Oral delivery daily dose | 40 | mg |
| CSF concentration of therapeutic | 500 | μM |
| Typical EC₅₀ value¹ | 200 | μM |
| Lifetime of photoactivated form of the drug (τₚₕₒₜₒ) | 15 | sec |

Illumination requirements

| | | |
|---|---|---|
| Required irradiance for rastered 1 cm³ volume | 2 | mW/mm² |
| activation | | |

| | |
|---|---|
| Per-voxel duty cycle for 1 cm³ volume activation | 0.1% |
| Photoactivated percentage achieved over 1 cm³ | 37% |

650-nm characteristics

| Focusing efficiency (15 mm depth) | 0.304% | |
|---|---|---|
| Irradiance at 1 mm³ focal point (15 mm depth) | 1.56 | mW/mm² |
| Focusing efficiency (30 mm depth) | 0.175% | |
| Irradiance at 1 mm³ focal point (30 mm depth) | 0.896 | mW/mm² |

850-nm characteristics

| Focusing efficiency (15 mm depth) | 0.468% | |
|---|---|---|
| Irradiance at 1 mm³ focal point (15 mm depth) | 2.4 | mW/mm² |
| Focusing efficiency (30 mm depth) | 0.264% | |
| Irradiance at 1 mm³ focal point (30 mm depth) | 1.35 | mW/mm² |

Even with these wavefront-shaped sources, the absorption that will consume heavily scattered photons needs to be overcome. This is done by maximizing the number of wavefront-corrected coherent sources that can be directed at the target, and their areal coverage; these sources are otherwise incoherent with respect to each other. Unfortunately, not all of the surface area of the scalp covered by the NIR-HEAD array can be illuminated because some area is needed for interconnect, electronics, and the detection array; this amounts to an illumination areal efficiency (as defined as the fraction of the area of the scalp covered by the chiplet array that receives illumination) of approximately 7% in the Phase 0 design, providing considerable opportunity for further improvement by reducing CMOS chiplet pitch in the array, incorporating more VCSELs per chiplet, and using waveguide trees for light distribution.

Table 2 shows some of the characteristics assumed for the photopharmaceutical being developed, including localized activation dose and light absorption characteristics. The per-molecule rate of photoconversion is given by kₚₕₒₜₒ=ΦσI/Eₚₕₒₜₒₙ, where I is the irradiance and Eₚₕₒₜₒₙ is the photon energy. kₚₕₒₜₒ is estimated to be 39.3 Hz, using the values of Φ and σ shown in Table 1 and assuming I=2 mW/mm² and Eₚₕₒₜₒₙ=2.34×10⁻¹⁹ J (corresponding to a wavelength of 850 nm). In addition, τₚₕₒₜₒ is the lifetime associated with the photoactivable form of the drug, which must be sufficiently short that any drug diffusing from the activated region will relax to its inactive form, preserving localization. The time-averaged fraction of molecules that will be photoactivated is given by

$$f \approx \frac{{k}_{photo} D}{{k}_{photo} D + {k}_{relax}} ,$$

where kᵣₑₗₐₓ=l/τₚₕₒₜₒ and D is the per voxel duty cycle that results from raster scanning to cover larger volumes as described below. A 1-mm³ focal spot can cover a 1000 mm³ volume with a D of 0.1% at each voxel. For this value of D, an activation fraction of f=37% can be achieved at I=2 mW/mm² irradiance. This activation fraction is close to the EC₅₀ values that can be expected with the assumptions of daily dosage and CSF concentrations shown in Table 2. This intensity can be reached by NIR-HEAD at 15 mm depth; at 30 mm depth, intensities about a factor of two lower are achieved. To reach>30% activation over a 1-cm³ volume at 30 mm will require increasing the available illumination at the skin surface by another factor of two; in all cases, this can be done while staying below ANSI limits because the average power will remain below 4 mW/mm² for any region. The present invention can reasonably achieve a factor of up to five by just increasing the illumination areal efficiency from the current 7% to 31.5% by incorporating four VCSELs per chip and reducing the chiplet pitch to accommodate a 12×12 array in the same area.

The requirements of achieving the peak 2-mW/mm² irradiance levels at the 1-mm² focus drive the entire patch geometry shown in FIG. 13a: a 48 mm×48 mm flexible patch design that contains an 8×8 array of 2.3 mm×2.3 mm complementary metal-oxide-semiconductor (CMOS) chiplets on a 6-mm pitch. Chiplets will be bonded into a polyimide flexible printed-circuit board (PCB) using solder-ball attach as shown in FIG. 14. For the rat, the present invention designs a 1 cm×1 cm array containing a 2×2 CMOS chiplet array on the same 6-mm pitch.

Each chiplet contains two independently controllable custom-fabricated vertical-cavity surface emitting lasers (VCSEL), delivering 5 mW of CW emission at a wavelength between 850 nm and 650 nm, depending on the precise photoactivation wavelength determined, at a wall-plug efficiency of approximately 30%. Spectral bandwidths (FWHM) for these designs are 2 nm. Microlenses, waveguide trees, metasurfaces, and SLMs allow the emission from individual VCSELs to be wavefront shaped and coupled into the scalp over a large surface area and over a wide angular range, maximizing the number of accessible high-transmission “eigenchannels” for light delivery deep into the scattering tissue and enabling light focusing in desired brain regions. Illumination of larger volumes of tissue is accomplished by raster scanning the 1-mm³ illumination volume using the 100-Hz frame rate available for the SLM described in § 4.3. Simulations (FIG. 9) yield the focusing efficiencies shown in Table 2, which imply that achieving the required irradiance at 30-mm depth at 650 nm will require a per-VCSEL output power of 5 mW for the 128 VCSELs of the NIR-HEAD device. The 5 mW of output power per VCSEL is reduced to 4 mW by the approximately 1.5 dB of loss expected in the waveguide distribution described in § 4.3. Other capabilities and requirements of the NIR-HEAD device are summarized below.

### Capabilities of the NIR-HEAD Device

### Measurement Capabilities

The measurement capabilities of the NIR-HEAD patch allow us to verify the optical coupling to the scalp and to measure the scattering and absorption properties of the tissue once the patch is positioned as part of a system calibration. This is done by using time-domain diffuse optical tomography (TD-DOT), building on capabilities that have already been developed for brain imaging^{6,7}. TD-DOT measurements are augmented by analysis of the temporal histograms and spatial patterns of back-scattered coherent photons and allow the focusing to the tuned with dynamic SLM adjustment (FIG. 13b) as described in more detail in § 4.5. To support this time-domain measurement capability, each chiplet contains an array of 20×20 single-photon avalanche diodes (SPADs) capable of detecting photons scattered back as well as the arrival time of these photons.

### Battery

For the highest total average optical output power for the array of 640 mW (CW operation at 5 mW peak power per VCSEL), an input electrical power of 2.1 W is required. Adding in the addition power overhead associated with the efficiency of the laser drivers, the predriver control, the TD-DOT functionality on some of the chiplets, and the wireless 802.11n interface to the device, the estimated total average power of the device is 2.7 W. For 10.8 W-h of battery storage, this amounts to approximately four hours of continuous operation. Lithium polymer (LiPo) batteries of a specific energy up to 265 W-h/kg can provide 10.8 W-h of storage in less than 40 g.

### Support Electronics

Digital control, power management, battery charging, decoupling capacitance, and the wireless interface are included on a 48 mm×25 mm rigid printed-circuit board that connects to the flexible array (as shown in FIG. 14). The flexible SLM also connects to this same rigid PCB. At approximately 750 W-h/L, a 10.8 W-h battery should require a volume of approximately 14.3 mL. If the entire area of the patch will be used for this mechanically flexible LiPo battery, this amounts to 17.5 mL (48 mm×73 mm×5 mm) at the 5-mm thickness requirement of the BAA.

### REFERENCES FOR DETAILED DESCRIPTION OF THE INVENTION SECTION

The following reference is hereby incorporated by references, in their entireties:
1. Finkelstein, E. A., D. S. Brown, L. A. Wrage, B. T. Allaire, and T. J. Hoerger, Individual and aggregate years-of-life-lost associated with overweight and obesity. Obesity (Silver Spring), 2010. 18(2): p. 333-9.
2. Finkelstein, E. A., J. G. Trogdon, J. W. Cohen, and W. Dietz, Annual medical spending attributable to obesity: payer-and service-specific estimates. Health Aff (Millwood), 2009. 28(5): p. w822-31.
3. Sadry, S. A. and D. J. Drucker, Emerging combinatorial hormone therapies for the treatment of obesity and T2DM. Nat Rev Endocrinol, 2013.
4. Drucker, D. J., S. I. Sherman, R. M. Bergenstal, and J. B. Buse, The safety of incretin-based therapies-review of the scientific evidence. J Clin Endocrinol Metab, 2011. 96(7): p. 2027-31.
5. Moore, P. W., K. Malone, D. Van Valkenburg, L. L. Rando, B. C. Williams, H. G. Matejowsky, S. Ahmadzadeh, S. Shekoohi, E. M. Cornett, and A. D. Kaye, GLP-1 Agonists for Weight Loss: Pharmacology and Clinical Implications. Advances in Therapy, 2023. 40(3): p. 723-742.
6. Yang, K., E. D. O'Cearbhaill, S. S. Liu, A. Zhou, G. D. Chitnis, A. E. Hamilos, J. Xu, M. K. S. Verma,
J. A. Giraldo, Y. Kudo, E. A. Lee, Y. Lee, R. Pop, R. Langer, D. A. Melton, D. L. Greiner, and J. M. Karp, A therapeutic convection-enhanced macroencapsulation device for enhancing β cell viability and insulin secretion. Proc Natl Acad Sci USA, 2021. 118(37).
7. Rorsman, P. and F. M. Ashcroft, Pancreatic β-Cell Electrical Activity and Insulin Secretion: Of Mice and Men. Physiol Rev, 2018. 98(1): p. 117-214.
8. Zeng, N., T. Jung, M. Sharma, G. Eichler, F. Fabbri, R. J. Cotton, E. Spinazzi, B. Youngerman, L. Carloni, and K. L. Shepard, A Wireless, Mechanically Flexible, 25_m-Thick, 65,536-Channel Subdural Surface Recording and Stimulating Microelectrode Array with Integrated Antennas, in Symposium on VLSI Circuit. 2023.
9. Chen, C., J. Yang, H. Wang, Z. Cao, S. Kananian, K. Chen, and A. S. Poon. A 90-μW penny-sized 1.2-gram wireless EEG recorder with 12-channel FDMA transmitter for month-long continuous mental health monitoring. in 2022 IEEE Symposium on VLSI Technology and Circuits (VLSI Technology and Circuits). 2022. IEEE.
10. Demir, A. F., Q. H. Abbasi, Z. E. Ankarali, E. Serpedin, and H. Arslan. Numerical characterization of in vivo wireless communication channels. in 2014 IEEE MTT-S International Microwave Workshop Series on RF and Wireless Technologies for Biomedical and Healthcare Applications (IMWS-Bio2014). 2014. IEEE.
11. Wentzloff, D. D., A. Alghaihab, and J. Im. Ultra-low power receivers for IoT applications: A review. in 2020 IEEE Custom Integrated Circuits Conference (CICC). 2020. IEEE.
12. Iguchi, S., T. Sakurai, and M. Takamiya, A low-power CMOS crystal oscillator using a stacked-amplifier architecture. IEEE Journal of Solid-State Circuits, 2017. 52(11): p. 3006-3017.
13. Xu, Z., N. Kimura, K. Okada, and M. Miyahara, Ultralow-power class-c complementary colpitts crystal oscillator. IEEE Solid-State Circuits Letters, 2020. 3: p. 274-277.
14. Pollmann, E. H., H. Yin, I. Uguz, A. Dubey, K. E. Wingel, J. S. Choi, S. Moazeni, Y. Gilhotra, V. A. Pavlovsky, A. Banees, V. Boominathan, J. Robinson, A. Veeraraghavan, V. A. Pieribone, B. Pesaran, and K. L. Shepard, Subdural CMOS optical probe (SCOPe) for bidirectional neural interfacing. bioRxiv, 2023: p. 2023.02.07.527500.
15. Wu, C.-Y., S.-H. Wang, and L.-Y. Tang, CMOS high-efficiency wireless battery charging system with global power control through backward data telemetry for implantable medical devices. IEEE Transactions on Circuits and Systems I: Regular Papers, 2020. 67(12): p. 5624-5635.
16. Pérez, P., F. Veirano, and F. Silveira, A compact lithium-ion battery charger for low-power applications. IEEE Transactions on Circuits and Systems II: Express Briefs, 2022. 69(3): p. 669-673.
17. Gilhotra, Y., H. Overhauser, H. Yin, E. Pollmann, G. Eichler, A. Cheng, T. Jung, N. Zeng, L. Carloni, and K. Shepard. A Wireless Subdural Optical Cortical Interface Device with 768 Co-Packaged Micro-LEDs for Fluorescence Imaging and Optogenetic Stimulation, in IEEE Custom Integrated Circuits Conference. 2024.
18. Moazeni, S., E. H. Pollmann, V. Boominathan, F. A. Cardoso, J. T. Robinson, A. Veeraraghavan, and K. L. Shepard. A Mechanically Flexible Implantable Neural Interface for Computational Imaging and Optogenetic Stimulation over 5.4×5.4 mm 2 FoV. in 2021 IEEE International Solid-State Circuits Conference (ISSCC). 2021. IEEE.
19. Moazeni, S., E. H. Pollmann, V. Boominathan, F. A. Cardoso, J. T. Robinson, A. Veeraraghavan, and K. L. Shepard. 19.2 A Mechanically Flexible Implantable Neural Interface for Computational Imaging and Optogenetic Stimulation over 5.4×5.4 mm<sup>2</sup> FoV. in 2021 IEEE International Solid-State Circuits Conference (ISSCC). 2021.

### REFERENCES FOR PHASES AND DISCUSSION SECTION

The following reference is hereby incorporated by references, in their entireties:
1 Robertson, S. D., Matthies, H. J. and Galli, A. A closer look at amphetamine-induced reverse transport and trafficking of the dopamine and norepinephrine transporters. Mol Neurobiol 39, 73-80 (2009).
2 Vellekoop, I. M. and Mosk, A. P. Focusing coherent light through opaque strongly scattering media. Optics letters 32, 2309-2311 (2007).
3 Fishkin, J. B., Gratton, E. and Mantulin, W. W. in Time-Resolved Spectroscopy and Imaging of Tissues. 122-135 (SPIE).
4 America, L. I. o. Vol. ANSI Z136.1-2022 (2022).
5 Cayce, J. M., Friedman, R. M., Chen, G., Jansen, E. D., Mahadevan-Jansen, A. and Roe, A. W. Infrared neural stimulation of primary visual cortex in non-human primates. Neuroimage 84, 181-190 (2014).
6 Moazeni, S., Renehan, K., Pollmann, E. H. and Shepard, K. L. in 2021 IEEE Custom Integrated Circuits Conference (CICC). 1-2 (IEEE).
7 Moazeni, S., Renehan, K., Pollmann, E. H. and Shepard, K. L. An Integrated-Circuit Node for High-Spatiotemporal Resolution Time-Domain Near-Infrared Diffuse Optical Tomography Imaging Arrays. IEEE Journal of Solid-State Circuits (2022).
8 Choquette, K. D. and Hou, H. Q. Vertical-cavity surface emitting lasers: Moving from research to manufacturing. Proceedings of the IEEE 85, 1730-1739 (1997).
9 Choquette, K. in Semiconductor Lasers 316-340 (Elsevier, 2013).
10 Choquette, K. D. Vertical-cavity surface-emitting lasers: light for the information age. MRS bulletin 27, 507-511 (2002).
11 Haghighi, N. and Lott, J. A. Electrically parallel three-element 980 nm VCSEL arrays with ternary and binary bottom DBR mirror layers. Materials 14, 397 (2021).
12 Shepard, K. L., Moazeni, S. and Renehan, K. (PCT/US2020/034682 2022).
13 Pollonini, L., Bortfeld, H. and Oghalai, J. S. PHOEBE: a method for real time mapping of optodes-scalp coupling in functional near-infrared spectroscopy. Biomedical optics express 7, 5104-5119 (2016).
14 Spinelli, L., Botwicz, M., Zolek, N., Kacprzak, M., Milej, D., Sawosz, P., Liebert, A., Weigel, U., Durduran, T. and Foschum, F. Determination of reference values for optical properties of liquid phantoms based on Intralipid and India ink. Biomedical optics express 5, 2037-2053 (2014).
Ayers, F., Grant, A., Kuo, D., Cuccia, D. J. and Durkin, A. J. in Design and Performance Validation of Phantoms Used in Conjunction with Optical Measurements of Tissue. 56-64 (SPIE).
16 Bellin, D. L., Sakhtah, H., Rosenstein, J. K., Levine, P. M., Thimot, J., Emmett, K., Dietrich, L. E. P. and Shepard, K. L. Integrated circuit-based electrochemical sensor for spatially resolved detection of redox-active metabolites in biofilms. Nat Commun 5 (2014).
17 Choi, J., Aklimi, E., Shi, C., Tsai, D., Krishnaswamy, H. and Shepard, K. L. Matching the Power, Voltage, and Size of Biological Systems: A nW-Scale, 0.023-Pulsed 33-GHz Radio Transmitter Operating From a 5 kT/q-Supply Voltage. Circuits and Systems I: Regular Papers, IEEE Transactions on 62, 1950-1958 (2015).
18 Field, R. M., Realov, S. and Shepard, K. L. A 100 fps, time-correlated single-photon-counting-based fluorescence-lifetime imager in 130 nm CMOS. IEEE Journal of Solid-State Circuits 49, 867-880 (2014).
19 Johnston, M. L., Kymissis, I. and Shepard, K. L. FBAR-CMOS Oscillator Array for Mass-Sensing Applications. Sensors Journal, IEEE 10, 1042-1047 (2010).
20 Lei, N., Ramakrishnan, S., Shi, P., Orcutt, J. S., Yuste, R., Kam, L. C. and Shepard, K. L. High-resolution extracellular stimulation of dispersed hippocampal culture with high-density CMOS multielectrode array based on non-Faradaic electrodes. Journal of neural engineering 8, 044003 (2011).
21 Lei, N., Watson, B. O., MacLean, J. N., Yuste, R. and Shepard, K. L. in Solid-State Circuits Conference, 2008. ISSCC 2008. Digest of Technical Papers. IEEE International. 148-603.
22 Levine, P. M., Ping, G., Levicky, R. and Shepard, K. L. Active CMOS Sensor Array for Electrochemical Biomolecular Detection. Solid-State Circuits, IEEE Journal of 43, 1859-1871 (2008).
23 Rosenstein, J., Lemay, S. G. and Shepard, K. L. Single-molecule bioelectronics. Wiley Interdisciplinary Reviews: Nanomedicine and Nanobiotechnology 7, 475-493 (2014).
24 Rosenstein, J., Ray, V., Drndic, M. and Shepard, K. L. in Solid-State Sensors, Actuators and Microsystems Conference (TRANSDUCERS), 2011 16th International. 874-877.
25 Sorgenfrei, S., Chiu, C. Y., Gonzalez, R. L., Yu, Y. J., Kim, P., Nuckolls, C. and Shepard, K. L. Label-free single-molecule detection of DNA-hybridization kinetics with a carbon nanotube field-effect transistor. Nature Nanotechnology 6, 125-131 (2011).
26 Tsai, D., John, E., Chari, T., Yuste, R. and Shepard, K. L. in 37th Annual International Conference of the IEEE EMBS (2015).
27 Warren, S. B., Vernick, S., Romano, E. and Shepard, K. L. Single-molecule field-effect transistor arrays with sub-microsecond temporal resolution. Nano Lett (2016).
28 Field, R. and Shepard, K. L. in Symposium on VLSI Circuits (Kyoto, Japan, 2013).
29 Field, R. M., Lary, J., Cohn, J., Paninski, L. and Shepard, K. L. A low-noise, single-photon avalanche diode in standard 0.13 μm complementary metal-oxide-semiconductor process. Applied Physics Letters 97, 211111-211111 (2010).
30 Choi, J., Taal, A. J., Pollmann, E. H., Lee, C., Kim, K., Moreaux, L. C., Roukes, M. L. and Shepard, K. L. A 512-Pixel, 51-kHz-Frame-Rate, Dual-Shank, Lens-Less, Filter-Less Single-Photon Avalanche Diode CMOS Neural Imaging Probe. IEEE Journal of Solid-State Circuits 54, 2957-2968 (2019).
31 Choi, J., Taal, A. J., Pollmann, E. H., Meng, W., Moazeni, S., Moreaux, L. C., Roukes, M. L. and Shepard, K. L. in 2019 IEEE Biomedical Circuits and Systems Conference (BioCAS). 1-4 (IEEE).
32 Gilhotra, Y., Overhauser, H., Yin, H., Pollmann, E., Eichler, G., Cheng, A., Jung, T., Zeng, N., Carloni, L. and Shepard, K. in IEEE Custom Integrated Circuits Conference.
33 Moazeni, S., Pollmann, E. H., Boominathan, V., Cardoso, F. A., Robinson, J. T., Veeraraghavan, A. and Shepard, K. A Mechanically Flexible, Implantable Neural Interface for Computational Imaging and Optogenetic Stimulation over 5.45. 4 mm 2 FoV. IEEE Transactions on Biomedical Circuits and Systems (2021).
34 Moazeni, S., Pollmann, E. H., Boominathan, V., Cardoso, F. A., Robinson, J. T., Veeraraghavan, A. and Shepard, K. L. in 2021 IEEE International Solid-State Circuits Conference (ISSCC). 288-290.
35 Pollmann, E. H., Yin, H., Uguz, I., Dubey, A., Wingel, K. E., Choi, J. S., Moazeni, S., Gilhotra, Y., Pavlovsky, V. A., Banees, A., Boominathan, V., Robinson, J., Veeraraghavan, A., Pieribone, V. A., Pesaran, B. and Shepard, K. L. Subdural CMOS optical probe (SCOPe) for bidirectional neural interfacing. bioRxiv, 2023.2002.2007.527500 (2023).
36 Yu, N. and Capasso, F. Flat optics with designer metasurfaces. Nature materials 13, 139-150 (2014).
37 Yu, N., Genevet, P., Kats, M. A., Aieta, F., Tetienne, J.-P., Capasso, F. and Gaburro, Z. Light Propagation with Phase Discontinuities: Generalized Laws of Reflection and Refraction. Science 334, 333-337 (2011).
38 Zhu, J., Udupa, A. and Goddard, L. L. Visualizable detection of nanoscale objects using anti-symmetric excitation and non-resonance amplification. Nature communications 11, 2754 (2020).
39 Zhu, J., Liu, Y., Yu, X., Zhou, R., Jin, J.-M. and Goddard, L. L. Sensing sub-10 nm wide perturbations in background nanopatterns using optical pseudoelectrodynamics microscopy (opem). Nano letters 19, 5347-5355 (2019).
40 Hwang, S.-W., Park, G., Edwards, C., Corbin, E. A., Kang, S.-K., Cheng, H., Song, J.-K., Kim, J.-H., Yu, S. and Ng, J. Dissolution chemistry and biocompatibility of single-crystalline silicon nanomembranes and associated materials for transient electronics. ACS nano 8, 5843-5851 (2014).
41 Bhaduri, B., Edwards, C., Pham, H., Zhou, R., Nguyen, T. H., Goddard, L. L. and Popescu, G. Diffraction phase microscopy: principles and applications in materials and life sciences. Advances in Optics and Photonics 6, 57-119 (2014).
42 Edwards, C., Bhaduri, B., Nguyen, T., Griffin, B. G., Pham, H., Kim, T., Popescu, G. and Goddard, L. L. Effects of spatial coherence in diffraction phase microscopy. Optics express 22, 5133-5146 (2014).
43 Kim, T., Zhou, R., Mir, M., Babacan, S. D., Carney, P. S., Goddard, L. L. and Popescu, G. White-light diffraction tomography of unlabelled live cells. Nature Photonics 8, 256-263 (2014).
44 Zhou, R., Edwards, C., Arbabi, A., Popescu, G. and Goddard, L. L. Detecting 20 nm wide defects in large area nanopatterns using optical interferometric microscopy. Nano letters 13, 3716-3721 (2013).
45 Edwards, C., Wang, K., Zhou, R., Bhaduri, B., Popescu, G. and Goddard, L. L. Digital projection photochemical etching defines gray-scale features. Optics Express 21, 13547-13554 (2013).
46 Richards, C. A., Ocier, C. R., Xie, D., Gao, H., Robertson, T., Goddard, L. L., Christiansen, R. E., Cahill, D. G. and Braun, P. V. Hybrid achromatic microlenses with high numerical apertures and focusing efficiencies across the visible. Nature communications 14, 3119 (2023).
47 Littlefield, A. J., Xie, D., Richards, C. A., Ocier, C. R., Gao, H., Messinger, J. F., Ju, L., Gao, J., Edwards, L. and Braun, P. V. Enabling high precision gradient index control in subsurface multiphoton lithography. ACS photonics 10, 3008-3019 (2023).
48 Ocier, C. R., Richards, C. A., Bacon-Brown, D. A., Ding, Q., Kumar, R., Garcia, T. J., Van De Groep, J., Song, J.-H., Cyphersmith, A. J. and Rhode, A. Direct laser writing of volumetric gradient index lenses and waveguides. Light: Science & Applications 9, 196 (2020).
49 Abdoudaey, G., Ren, K. and Hielscher, A. H. Optical tomography as a constrained optimization problem. Inverse Problems 21, 1507-1530 (2005).
50 Altoe, M. L., Kalinsky, K., Marone, A., Kim, H. K., Guo, H., Hibshoosh, H., Tejada, M., Crew, K. D., Accordino, M. K., Trivedi, M. S., Hershman, D. L. and Hielscher, A. H. Changes in Diffuse Optical Tomography Images During Early Stages of Neoadjuvant Chemotherapy Correlate with Tumor Response in Different Breast Cancer Subtypes. Clinical Cancer Research 27, 1949-1957 (2021).
51 Altoe, M. L., Marone, A., Kim, H. K., Kalinsky, K., Hershman, D. L., Hielscher, A. H. and Ha, R. S. Diffuse optical tomography of the breast: a potential modifiable biomarker of breast cancer risk with neoadjuvant chemotherapy. Biomedical Optics Express 10, 4305-4315 (2019).
52 Bluestone, A. Y., Stewart, M., Lei, B., Kass, I. S., Lasker, J., Abdoulaev, G. S. and Hielscher, A. H. Three-dimensional optical tomographic brain imaging in small animals, Part I: Hypercapnia. Journal of Biomedical Optics 9, 1046-1062 (2004).
53 Dohm, J. C., Lottaz, C., Borodina, T. and Himmelbauer, H. Substantial biases in ultra-short read data sets from high-throughput DNA sequencing. Nucleic Acids Res. 36, e105 (2008).
54 Flexman, M., Kim, H., Stoll, R., Khalil, M., Fong, C. and Hielscher, A. A wireless 54 handheld probe with spectrally constrained evolution strategies for diffuse optical imaging of tissue. Review of Scientific Instruments 83, 033108 (2012).
55 Flexman, M. L., Khalil, M. A., Kim, H. K., Fong, C. J., Hielscher, A. H., Al Abdi, R., Barbour, R. L., Desperito, E. and Hershman, D. L. Digital optical tomography system for dynamic breast imaging. Journal of biomedical optics 16, 076014 (2011).
56 Flexman, M. L., Kim, H. K., Gunther, J. E., Lim, E., Alvarez, M. C., Desperito, E., Kalinsky, K., Hershman, D. L. and Hielscher, A. H. Optical biomarkers for breast cancer derived from dynamic diffuse optical tomography. Journal of biomedical optics 18, 096012 (2013).
57 Gibson, A. P. and et al. Recent advances in diffuse optical imaging. Physics in Medicine and Biology 50, R1 (2005).
58 Hielscher, A. H. Optical tomographic imaging of small animals. Current Opinion in Biotechnology 16, 79-88 (2005).
59 Hielscher, A. H., Alcouffe, R. E. and Barbour, R. L. Comparison of finite-difference transport and diffusion calculations for photon migration in homogenous and heterogeneous tissues. Phys. Med. Biol 43, 1285-1302 (1998).
60 Hielscher, A. H., Bluestone, A. Y., Abdoulaev, G. S., Klose, A. D., Lasker, J., Stewart, M., Netz, U. and Beuthan, J. Near-infrared diffuse optical tomography. Disease Markers 18, 313-337 (2002).
61 Hielscher, A. H., Kim, H. K., Montejo, L. D., Blaschke, S., Netz, U. J., Zwaka, P. A., Illing, G., Muller, G. A. and Beuthan, J. Frequency-domain optical tomographic imaging of arthritic finger joints. IEEE transactions on medical imaging 30, 1725-1736 (2011).
62 Hielscher, A. H., Klose, A. and Beuthan, J. Evolution strategies for optical tomographic characterization of homogeneous media. Opt. Express 9, 507-518 (2000).
63 Hielscher, A. H., Klose, A. D. and Hanson, K. M. Gradient-based iterative image reconstruction scheme for time-resolved optical tomography. IEEE Trans. Med. Imag. 18, 262-271 (1999).
64 Hielscher, A. H., Klose, A. D., Scheel, A., Moa-Anderson, B., Backhaus, M., Netz, U. and Beuthan, J. Sagittal laser optical tomography for imaging of rheumatoid finger joints. Physics in Medicine and Biology 49, 1147-1163 (2004).
65 Hoi, J., Kim, H., Fong, C., Zweck, L. and Hielscher, A. Non-contact dynamic diffuse optical tomography imaging system for evaluating lower extremity vasculature. Biomedical optics express 9, 5597-5614 (2018).
66 Jacques, S. L. and Hielscher, A. H. Discriminating the size and density of objects within tissue: frequency-domain versus steady-state measurements. SPIE Proc. 2389, 228-239 (1995).
67 Jia, J., Kim, H. K. and Hielscher, A. H. Fast linear solver for radiative transport equation with multiple right hand sides in diffuse optical tomography. Journal of Quantitative Spectroscopy and Radiative Transfer 167, 10-22 (2015).
68 Khalil, M., Kim, H., Hoi, J., Kim, I., Dayal, R., Shrikhande, G. and Hielscher, A. Detection of peripheral arterial disease within the foot using vascular optical tomographic imaging: a clinical pilot study. European Journal of Vascular and Endovascular Surgery 49, 83-89 (2015).
69 Kim, H. K., Flexman, M., Yamashiro, D. J., Kandel, J. J. and Hielscher, A. H. PDE-constrained multispectral imaging of tissue chromophores with the equation of radiative transfer. Biomedical optics express 1, 812-824 (2010).
70 Kim, H. K. and Hielscher, A. H. A diffusion-transport hybrid method for accelerating optical tomography. Journal of Innovative Optical Health Sciences 3, 293-305 (2010).
71 Kim, H. K., Lee, J. H. and Hielscher, A. H. PDE-constrained fluorescence tomography with the frequency-domain equation of radiative transfer. IEEE Journal of Selected Topics in Quantum Electronics 16, 793-803 (2010).
72 Kim, H. K., Lee, J. H., Lim, E. and Hielscher, A. H. in Clinical and Translational Biophotonics. JTh3A. 12 (Optica Publishing Group).
73 Kim, H. K., Montejo, L. D., Jia, J. and Hielscher, A. H. Frequency-domain optical tomographic image reconstruction algorithm with the simplified spherical harmonics (SP3) light propagation model. International Journal of Thermal Sciences 116, 265-277 (2017).
74 Kim, H. K., Tucker, N., Debernardis, F. and Hielscher, A. H. in Cancer Imaging and Therapy. JTu3A. 26 (Optica Publishing Group).
75 Kim, H. K., Zhao, Y., Raghuram, A., Veeraraghavan, A., Robinson, J. and Hielscher, A. H. Ultrafast and Ultrahigh-Resolution Diffuse Optical Tomography for Brain Imaging with Sensitivity Equation based Noniterative Sparse Optical Reconstruction (SENSOR). Journal of Quantitative Spectroscopy and Radiative Transfer 276, 107939 (2021).
76 Kim, S. H., Montejo, L. and Hielscher, A. Diagnostic Evaluation of Rheumatoid Arthritis (RA) in Finger Joints Based on the Third-Order Simplified Spherical Harmonics (SP3) Light Propagation Model. Applied Sciences 12, 6418 (2022).
77 Kim, Y., Marone, A., Tang, W., Gartshteyn, Y., Kim, H. K., Askanase, A. D., Kymissis, I. and Hielscher, A. H. Flexible optical imaging band system for the assessment of arthritis in patients with systemic lupus erythematosus. Biomedical Optics Express 12, 1651-1665 (2021).
78 Klose, A., Hielscher, A., Hanson, K. and Beuthan, J. Two- and three-dimensional optical tomography of finger joints for diagnostics of rheumatoid arthritis. Proc. SPIE 3566, 151-159 (1998).
79 Klose, A. D. and Hielscher, A. H. Iterative reconstruction scheme for optical tomography based on the equation of radiative transfer. Med Phys. 26, 1698-1707 (1999).
80 Klose, A. K. and Hielscher, A. H. Diffuse optical tomography with the equation of radiative transfer. International Journal for Numerical Methods in Heat and Fluid Flow (accepted for publication)
81 Klose, A. K. and Hielscher, A. H. Fluorescence tomography with the equation of radiative transfer for molecular imaging. Opt Lett 28, 1019-1021 (2003).
82 Lasker, J. M., Fong, C. J., Ginat, D. T., Dwyer, E. and Hielscher, A. H. Dynamic Optical Imaging of Vascular and Metabolic Reactivity in Rheumatoid Joints. J. Biomed. Opt. 12, 052001 (2007).
83 Lasker, J. M., Masciotti, J., Schoenecker, M., Schmitz, C. and Hielscher, A. H. Digital-signal-processor-based dynamic optical tomography imaging system. Rev. Sci. Instrum. 78, 083706 (2007).
84 Lee, J., Poschinger, T., Hernandez, S., Huang, J., Johung, T., Kandel, J., Yamashiro, D. J. and Hielscher, A. H. Dynamic Fluorescence Imaging for the Detection of Vascular Changes in Anti-Angiogenic Drug Therapy. Biomedical Optics, OSA Technical Digest (Optical Society of America) (2010).
85 Lee, J., Poschinger, T., Hernandez, S. L., Huang, J., Johung, T., Kandel, J., Yamashiro, D. J. and Hielscher, A. H. in Bioengineering Conference, Proceedings of the 2010 IEEE 36th Annual Northeast. 1-2.
86 Lee, J. H., Kim, H. K., Chandhanayingyong, C., Lee, F. Y.-I. and Hielscher, A. H. Non-contact small animal fluorescence imaging system for simultaneous multi-directional angular-dependent data acquisition. Biomedical optics express 5, 2301-2316 (2014).
87 Lim, E. A., Gunther, J. E., Kim, H. K., Flexman, M., Hibshoosh, H., Crew, K., Taback, B., Campbell, J., Kalinsky, K. and Hielscher, A. Diffuse optical tomography changes correlate with residual cancer burden after neoadjuvant chemotherapy in breast cancer patients. Breast cancer research and treatment 162, 533-540 (2017).
88 Masciotti, J. M., Lasker, J. M. and Hielscher, A. H. Digital lock-in detection for discriminating multiple modulation frequencies with high accuracy and computational efficiency. IEEE Transactions on Instrumentation and Measurement 57, 182-189 (2007).
89 Montejo, L. D., Jia, J., Kim, H. K., Netz, U. J., Blaschke, S., Müller, G. A. and Hielscher, A. H. Computer-aided diagnosis of rheumatoid arthritis with optical tomography, Part 1: feature extraction. Journal of biomedical optics 18, 076001 (2013).
90 Montejo, L. D., Klose, A. D. and Hielscher, A. H. Implementation of the equation of radiative transfer on block-structured grids for modeling light propagation in tissue. Biomedical optics express 1, 861-878 (2010).
91 Mourant, J. R., Hielscher, A. H., Eick, A. A., Johnson, T. M. and Freyer, J. P. Evidence of intrinsic differences in the light scattering properties of tumorigenic and nontumorigenic cells. Cancer Cytopath 84, 366-374 (1998).
92 Opitz, A., Falchier, A., Yan, C. G., Yeagle, E. M., Linn, G. S., Megevand, P., Thielscher, A., Deborah, A. R., Milham, M. P., Mehta, A. D. and Schroeder, C. E. Spatiotemporal structure of intracranial electric fields induced by transcranial electric stimulation in humans and nonhuman primates. Sci Rep 6, 31236 (2016).
93 Ren, K., Abdoulaev, G. S., Bal, G. and Hielscher, A. H. Frequency-domain optical tomography based on the equation of radiative transfer. SIAM Journal on Scientific Computing 28, 1463-1489 (2006).
94 Schell, A. K., Backhaus, M., Klose, A. D., Moa-Anderson, B., Netz, U., Hermann, K. G., Beuthan, J., Mueller, G. A., Burmester, G. R. and Hielscher, A. H. First clinical evaluation of sagittal laser optical tomography for detection of synovitis in arthritic finger joints. Annals of the Rheumatic Diseases 64, 239-245 (2005).
95 Wang, F., Kim, S. H., Zhao, Y., Raghuram, A., Veeraraghavan, A., Robinson, J. and Hielscher, A. H. High-Speed Time-Domain Diffuse Optical Tomography With a Sensitivity Equation-Based Neural Network. IEEE Transactions on Computational Imaging (2023).
96 Matar, A. J., Sachs, D. H. and Duran-Struuck, R. The MHC-characterized Miniature Swine: Lessons Learned From a 40-Year Experience in Transplantation. Transplantation 106, 928-937 (2022).
97 Fortin, S. M. and Roitman, M. F. Central GLP-1 receptor activation modulates cocaine-evoked phasic dopamine signaling in the nucleus accumbens core. Physiology & Behavior 176, 17-25 (2017).
98 Fortin, S., Cone, J., Ng-Evans, S., Mccutcheon, J. and Roitman, M. Sampling phasic dopamine signaling with fast-scan cyclic voltammetry in awake, behaving rats. Current protocols in neuroscience 70, 7.25. 21-27.25. 20 (2015).
99 Fortin, S. M. and Roitman, M. F. Physiological state tunes mesolimbic signaling: Lessons from sodium appetite and inspiration from Randall R. Sakai. Physiology & behavior 178, 21-27 (2017).
100 Cone, J. J., Fortin, S. M., McHenry, J. A., Stuber, G. D., Mccutcheon, J. E. and Roitman, M. F. Physiological state gates acquisition and expression of mesolimbic reward prediction signals. Proceedings of the National Academy of Sciences 113, 1943-1948 (2016).
101 Fortin, S. M., Chartoff, E. H. and Roitman, M. F. The aversive agent lithium chloride suppresses phasic dopamine release through central GLP-1 receptors. Neuropsychopharmacology 41, 906-915 (2016).
102 Fortin, S. M. and Roitman, M. F. Challenges to body fluid homeostasis differentially recruit phasic dopamine signaling in a taste-selective manner. Journal of Neuroscience 38, 6841-6853 (2018).
103 Fortin, S. M., Chen, J. C., Petticord, M. C., Ragozzino, F. J., Peters, J. H. and Hayes, M. R. The locus coeruleus contributes to the anorectic, nausea, and autonomic physiological effects of glucagon-like peptide-1. Science Advances 9, eadh0980 (2023).
104 Fortin, S. M., Lipsky, R. K., Lhamo, R., Chen, J., Kim, E., Borner, T., Schmidt, H. D. and Hayes, M. R. GABA neurons in the nucleus tractus solitarius express GLP-1 receptors and mediate anorectic effects of liraglutide in rats. Science Translational Medicine 12, eaay8071 (2020).
105 Kanoski, S. E., Fortin, S. M., Ricks, K. M. and Grill, H. J. Ghrelin signaling in the ventral hippocampus stimulates learned and motivational aspects of feeding via PI3K-Akt signaling. Biological Psychiatry 73, 915-923 (2013).
106 Kanoski, S. E., Hayes, M. R., Greenwald, H. S., Fortin, S. M., Gianessi, C. A., Gilbert, J. R. and Grill, H. J. Hippocampal leptin signaling reduces food intake and modulates food-related memory processing. Neuropsychopharmacology 36, 1859-1870 (2011).
107 Kanoski, S. E., Rupprecht, L. E., Fortin, S. M., De Jonghe, B. C. and Hayes, M. R. The role of nausea in food intake and body weight suppression by peripheral GLP-1 receptor agonists, exendin-4 and liraglutide. Neuropharmacology 62, 1916-1927 (2012).
108 Kanoski, S. E., Alhadeff, A. L., Fortin, S. M., Gilbert, J. R. and Grill, H. J. Leptin signaling in the medial nucleus tractus solitarius reduces food seeking and willingness to work for food. Neuropsychopharmacology 39, 605-613 (2014).

1. A device for delivering near-infrared (NIR) light into a tissue of a subject, wherein the device comprises:
(a) one or more batteries;
(b) one or more Complementary Metal-Oxide-Semiconductor (CMOS) chiplets;
(c) one or more rigid electronics boards;
(d) one or more flexible polyimide boards;
(e) one or more laser devices;
(f) one or more metasurface chiplets; and
(g) one or more spatial light modulators (SLMs).

2. The device of claim 1, wherein the device
(a) focuses the NIR light into the tissue; preferably, the distance between the focal point of the focused NIR light and the tissue is from 1 mm to 50 mm;
more preferably, from 10 mm to 40 mm; more preferably, from 15 mm to 40 mm; more preferably, from 15 mm to 30 mm; and
(b) detects backscattered photons.

3. The device of claim 1, wherein the battery is lithium battery; preferably, lithium polymer (LiPO) battery.

4. The device of claim 1, wherein the device comprises 8×8 CMOS chiplets; preferably each chiplet comprises two laser devices.

5. The device of claim 4, wherein the
(a) CMOS chiplets contain one or more software drivers;
(b) CMOS chiplets comprise single-photon avalanche diodes (SPADs); preferably, each CMOS chiplet comprises an array of 20×20 SPADs; preferably, the array of 20×20 SPADs measures the time-of-flight (Tof) of backscattered photons; and/or
(c) CMOS chiplets are mounted to the flexible polyimide board.

6. The device of claim 1, wherein the rigid electronics board comprises one of more components for signal processing, digital control, power management, battery charging, decoupling capacitance, and wireless interface;
preferably, the rigid electronics boards comprise Field-Programmable Gate Arrays (FPGAs) and a platform comprising a processor and a memory;
more preferably, the rigid electronics board is a printed-circuit board (PCB);
more preferably, the rigid electronic board connects to the flexible polyimide board and connects to the SLMs;
more preferably, one or more zero-insertion-force (ZIF) connectors connect the rigid electronics board to the SLMs.

7. The device of claim 1, wherein the flexible polyimide board is flexible PCB; preferably, the CMOS chiplets are mounted to the flexible PCB.

8. The device of claim 1, wherein the laser devices are vertical cavity surface emitting lasers (VCSELs); preferably, VCSELs employing AlₓGa₁₋ₓAs/Al_{y}Ga_{1-y}As distributed Bragg reflectors;
more preferably, VCSELs comprising an active region comprising multiple quantum wells of In_{y}Ga_{1-y}As/Al₁₋ₓGaₓAs or InGaAs/GaAs₁₋ₓPₓ. VCSELs;
more preferably, each VCSEL has an output power from 1 to 20 mW; preferably from 1 to 10 mW; more preferably 5 mW;
more preferably, each VCSEL emits a laser at a wavelength from 600 to 900 nm;
preferably from 650 to 850 nm; more preferably, a wavelength at 650 nm, 700 nm, 750 nm, 800 nm, or 850 nm.

9. The device of claim 1, wherein each of the metasurface chiplets further comprises:
(a) a lens;
(b) a network of optical waveguides (waveguide tree); and
(c) metalenses;
preferably, wherein when an input laser passes through the lens, the waveguide tree evenly splits the laser into multiple output emissions over an area;
more preferably, the waveguide tree evenly splits the laser into 16×16 output emissions over an area of 1 mm×1 mm;
more preferably, the metalenses then convert the 16×16 output emissions into 64×96 high Numerical Aperture (NA) focal spots as inputs for the SLM.

10. The device of claim 9, wherein the metasurface comprises 2D arrays of silicon nano-pillars;
preferably the nano-pillars have a height of 700 nm and diameters from 100 nm to 290 nm.

11. The device of claim 1, wherein the SLMs shape the wavefront of emission from each of the VCSELs;
preferably, the SLMs produce a phase shift of up to about 0.67π or about 120° for the 850-nm light at the normal incident angle;
more preferably, the SLM comprises a liquid crystal layer (LCD) encapsulated by flexible and ultra-thin membranes.

12. The device of claim 1, wherein
(a) the device delivers a power of at least 4 mW per mm² of the subject's tissue;
(b) the subject is a mammal; preferably human;
(c) the tissue is brain tissue; and/or
(d) the device delivers near-infrared (NIR) light up to about 3 cm below the surface of the skull of the subject.

13. An wearable device for the focused direction of light into tissue, comprising:
an array of laser devices; and
at lease one of the metasurfaces and/or spatial light modulators (SLMs) arranged to facilitate a wavefront shaping for each of the laser devices of the array.

14. The device of claim 13, wherein the laser devices are solid-state lasers.

15. The device of claim 13, wherein the lasers devices are vertical-cavity surface-emitting lasers.

16. The device of claim 13, wherein the metasurfaces and/or SLMs are arranged to generate one or more diffraction limits

17. The device of claim 13, wherein the laser devices are provided as nodes, wherein each of the nodes comprises chips of complementary metal-oxide-semiconductor (CMOS) integrated circuits, and wherein the laser devices are mounted on the chips in each node of the nodes.

18. A method for providing a wearable device that can facilitate a focused direction of a light into a tissue, the method comprising
(a) providing an array of laser devices, and a configuration that includes at least one metasurfaces or spatial light modulators;
(b) causing a wavefront shaping for each of the laser devices using the at least one of the metasurfaces or the spatial light modulators,
or
a method of delivering focused light into a predetermined area of a tissue in a subject, wherein the method comprises the subject wearing a device described herein;
or
a method of imaging a part of a subject, wherein the method comprises the subject wearing a device described herein; preferably, the part of the subject is the subject's brain, and delivering focused light to the part of the subject and collecting a signal therefrom so as to image the part of the subject;
or
a method of activating one or more photoswitch molecules in a subject, wherein the method comprises the subject wearing a device described herein, wherein the device delivers a focused direction of light into the subject's tissue that activates the one or more photoswitch molecules; preferably, the one or more photoswtich molecules comprise a photo-pharmaceutical; more preferably, photoactivatable dextroamphetamines.
